(19) United States
(12) Patent Application Publication
(10) Pub. No.: US 2025/0236660 A1
(43) Pub. Date: Jul. 24, 2025

(54) THERAPEUTIC COMBINATIONS AND METHODS TO TREAT LONG COVID

(71) Applicant: Rohan Dixit, San Francisco, CA (US)

(72) Inventor: Rohan Dixit, San Francisco, CA (US)

(21) Appl. No.: 19/176,141

(22) Filed: Apr. 10, 2025

Related U.S. Application Data
(63) Continuation-in-part of application No. PCT/US23/34865, filed on Oct. 10, 2023.
(60) Provisional application No. 63/414,662, filed on Oct. 10, 2022.

(51) Int. Cl. C07K 16/10; A61K 31/403; A61K 31/427; A61K 31/7072; A61K 39/00; A61K 45/06; A61P 31/14

(52) U.S. Cl. CPC C07K 16/1003; A61K 31/403; A61K 31/427; A61K 31/7072; A61K 39/00; A61K 45/06; A61P 31/14

(57) ABSTRACT

The disclosure relates in some aspects to methods of treating a subject having post-acute sequelae of COVID-19 (PASC), sometimes also known as “long COVID.” Also provided are pharmaceutical combinations for treating a subject having PASC, such as using one or more antiviral agents and one or more antibodies, as well as kits used with disclosed pharmaceutical combinations and in disclosed methods.

### CROSS-REFERENCE

This is a U.S. continuation-in-part application under § 111(a) of International Appl. No. PCT/US23/34865, filed Oct. 10, 2023, which claimed priority under PCT Article 8(1) and Rule 4.10 to U.S. Provisional Appl. No. 63/414,662, filed Oct. 10, 2022; each incorporated by reference for all purposes as if fully set forth herein.

### FIELD OF THE INVENTION

This disclosure relates generally to the medical treatment of viral infections. In certain aspects, it relates to methods of treating a subject having post-acute sequelae of COVID-19 (PASC). In some aspects, it further relates to pharmaceutical combinations and kits thereof, which can be used in disclosed methods.

### BACKGROUND OF THE INVENTION

COVID-19 is a contagious viral disease caused by infection of the SARS-CoV-2 virus. Infection cases of the SARS-CoV-2 virus and deaths related to COVID-19 complications have surpassed the millions, and COVID-19 is a significant global issue. Post-acute sequelae of COVID-19 (PASC), sometimes called “long COVID-19” or “long COVID,” is a continuation or emergence of symptoms weeks or longer after the initial SARS-CoV-2 infection. These symptoms may include fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, stomach pain, and numerous others.

Due to the recency of COVID-19 itself, the incidence, diagnosis, and prognosis of PASC have not been well explored. More importantly, effective treatments for post-acute sequelae of COVID-19 also remain elusive, and the continued prevalence of PASC leaves an unmet need to prevent and treat its symptoms.

### INCORPORATION BY REFERENCE

Each cited patent, publication, and non-patent literature is hereby incorporated by reference in its entirety, as if each was incorporated by reference individually and fully set forth herein. However, no such citation should be construed as an admission that a reference is from an analogous art or is directly applicable to the invention, nor should any citation be construed as an admission that a document or any underlying information, in any jurisdiction, is prior art or is part of the common general knowledge in the art.

### BRIEF SUMMARY OF THE INVENTION

The following is a simplified summary of some embodiments of the invention in order to provide a basic understanding thereof. It is not an extensive overview, nor is it intended to identify every key or critical element of the invention or to delineate the complete scope of the invention. Its sole purpose is to present some exemplary embodiments in a simplified form as a prelude to the more detailed description below.

In a first aspect, provided are methods of treating a subject having post-acute sequelae of COVID-19 (PASC), comprising administering to a subject an effective amount of a combination of: i) a first antiviral agent, or a pharmaceutically acceptable salt thereof; ii) optionally, a second antiviral agent, or a pharmaceutically acceptable salt thereof; iii) a first antibody; and iv) optionally, a second antibody.

In another aspect, provided are pharmaceutical kits for treating a subject having post-acute sequelae of COVID-19 (PASC), comprising: i) a first antiviral agent, or a pharmaceutically acceptable salt thereof; ii) optionally, a second antiviral agent, or a pharmaceutically acceptable salt thereof; iii) a first antibody; and iv) optionally, a second antibody.

In embodiments, the first antiviral agent is a protease inhibitor. In embodiments, the first antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

In embodiments, the first antibody is a monoclonal antibody. In embodiments, the first antibody is an Immunoglobulin G (IgG) antibody. In embodiments, the first antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, Anti-Endothelin Receptor B Antibody (AER002), and durvalumab.

In embodiments, the second antiviral agent is a protease inhibitor. In embodiments, the second antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

In embodiments, the second antibody is a monoclonal antibody. In embodiments, the second antibody is an IgG antibody. In embodiments, the second antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, AER002, and durvalumab.

In embodiments, the disclosed methods further comprise administering an effective amount of an additional active agent. In embodiments, the disclosed pharmaceutical kits further comprise an additional active agent. In embodiments, the additional active agent is any of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, monoamine oxidase inhibitors, empathogens, entactogens, entheogens, psychedelics, dissociatives, nootropics, euphorics, sedatives, stimulants, and vitamins.

In embodiments, the additional active agent is any of an uncoating inhibitor, entry inhibitor, protease inhibitor, immunostimulatory agent, latency reversal agent, reverse transcriptase, reverse transcriptase inhibitor, integrate inhibitor, nucleoside analogue, RNA polymerase inhibitor, viral uncapping agent, neuraminidase inhibitor, fusion inhibitor, interferon, nucleoside reverse transcriptase inhibitor, or non-nucleoside reverse transcriptase inhibitor. In embodiments, the additional active agent is an immunostimulatory agent, a COVID-19 vaccine, an additional agent having antiviral activity, or an additional antibody. In embodiments, the additional active agent is an additional agent having antiviral activity. In embodiments, the additional agent having antiviral activity is MIR 19® (siR-7-EM/KK-46), baicalein, peginterferon lambda, metformin, ursodeoxycholic acid (UCDA), TVGN-489, LAU-7b, sabizabulin, azithromycin, ivermectin, opanganib, baricitinib, mycophenolate, APN01, epigallocatechin gallate (EGCG), diphenhydramine, quercetin, Qing-Fei-Pai-Du-Tang (QFPDT), Lianhua Qingwen (LQ), Taiwan Chingguan Yihau (NRICM101), Jing Si herbal drink (JSHD), nicotine, lactoferrin, nattokinase, or ubiquinone.

In embodiments, the first antiviral agent is nirmatrelvir, sofosbuvir, or ritonavir, including any as a pharmaceutically acceptable salt thereof. In embodiments, the first antiviral agent is sofosbuvir, or a pharmaceutically acceptable salt thereof. In embodiments, the first antiviral agent is nirmatrelvir and the second antiviral agent is ritonavir, including either or both as a pharmaceutically acceptable salt thereof.

In embodiments, the first antibody is tixagevimab or cilgavimab. In embodiments, the first antibody is tixagevimab and the second antibody is cilgavimab.

In embodiments, the first antiviral agent is nirmatrelvir, or a pharmaceutically acceptable salt thereof; and the second antiviral agent is ritonavir, or a pharmaceutically acceptable salt thereof; the first antibody is tixagevimab; and the second antibody is cilgavimab. In embodiments, the first antiviral agent is sofosbuvir, or a pharmaceutically acceptable salt thereof; the first antibody is tixagevimab; and the second antibody is cilgavimab.

In embodiments, the first antibody and the second antibody are administered in the same composition, i.e., in a single pharmaceutical composition (i.e., co-formulated as a single pharmaceutical composition). In embodiments, the first antibody and the second antibody are comprised in the same composition, i.e., as a single pharmaceutical composition comprising both the first antibody and the second antibody. In other embodiments, the first antiviral agent and the second antiviral agent are administered in the same composition. In other embodiments, the first antiviral agent and the second antiviral agent are comprised in the same composition. In embodiments generally, any one or more of the disclosed agents may be administered in the same composition, or comprised in the same composition.

In embodiments, the method results in a reduction in the incidence or severity of at least one symptom of PASC. In embodiments, the symptom of PASC is a neurological symptom, a cardiovascular symptom, a pulmonary symptom, a musculoskeletal symptom, an endocrinological symptom, a dermal symptom, or a kidney symptom.

In embodiments, the neurological symptom is selected from the group consisting of sleep disorders, chronic headaches, olfactory disorders, taste disorders, brain fog, memory loss, decreased concentration, depression, anxiety, post-traumatic stress disorder (PTSD), dizziness, vertigo, tinnitus, hearing loss, instability, delirium, hallucinations, small fiber neuropathy, postural tremor, chronic pain, neurodegeneration, and myalgia.

In embodiments, the cardiovascular symptom is selected from the group consisting of nonspecific chest pain, tightness in the chest, palpitations, tachycardia, conduction disturbances, rhythm disorders, orthostatic hypotension, vasovagal syncope, postural orthostatic tachycardia syndrome (POTS), phlebitis, and thrombophlebitis.

In embodiments, the pulmonary symptom is selected from the group consisting of dyspnea, persistent cough, wheezing, worsening of asthma, decreased pulmonary diffusion capacity, persistent abnormal imaging findings, pleuritis, cough, sleep apnea, and sore throat.

In embodiments, the musculoskeletal symptom is selected from the group consisting of arthritis, back pain, abnormal gait and joint pain.

In embodiments, the endocrinological symptom is selected from the group consisting of impaired glucose metabolism, subacute thyrotoxicosis, Hashimoto's disease, Graves' disease, and dyslipidemia.

In embodiments, the dermal symptom is selected from the group consisting of eruption, urticaria, skin lesion, telogen effluvium, nail changes, erythema, and trichodynia.

In embodiments, the kidney symptom is selected from the group consisting of decreased globural filtration rate, urinary incontinence, and microscopic hematuria.

In embodiments, the symptom of PASC is selected from the group consisting of fever, chills, muscle ache, body ache, chest pain, stomach pain, nausea, vomiting, diarrhea, gastroesophageal reflux, stress, swelling, bleeding, weight loss, weight gain, abdominal pain, constipation, pain in extremities, paresthesia, peripheral edema, itchiness, lymphadenopathy, and edema.

In embodiments, the incidence of a symptom of PASC is reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%, as compared to the incidence of the symptom prior to treatment with the method.

In embodiments, the severity of a symptom PASC is reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%, as compared to the severity of the symptom prior to treatment with the method.

In embodiments, the method results in the serum concentration of a PASC biomarker returning to within about 50%, 40%, 30%, 20%, 10%, 5%, or 1% of a baseline concentration.

In embodiments, the PASC biomarker is a neurological biomarker. In embodiments, the neurological biomarker is GDNF, GFAP, NGF-β, NFL, NT-3, or pGFAP/pNFL.

In embodiments, the PASC biomarker is a cardiovascular biomarker. In embodiments, the cardiovascular biomarker is Ang-2, Col1A2, Col3A1, D-dimer, ESR, ET-1, Factor VIII:C, Hemoglobin, MMP-1, MMP-9, MPO, NO, PDGF-BB, SICAM-1, sTM, sVEGFR, sVCAM-1, VEGF, VWF:Ag, or VWF:pp.

Also provided is the use of the pharmaceutical kit of any of the foregoing embodiments for treating PASC. Also provided is the use of the pharmaceutical kit of any of the foregoing embodiments for the manufacture of a medicament for treating PASC.

The foregoing has outlined broadly some pertinent features of certain exemplary embodiments of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific formulations and methods may be readily utilized as a basis for modifying or designing other formulations and methods for carrying out the same purposes of the disclosure. It should be also realized that such equivalent formulations and methods do not depart from the spirit and scope of the invention as set forth in the claims. Hence, this summary is made with the understanding that it will be considered as a brief and general synopsis of only some of the objects and embodiments herein, is provided solely for the benefit and convenience of the reader, and is not intended to limit in any manner the scope, or range of equivalents, to which the claims are lawfully entitled. It also will be appreciated that the headings herein are utilized only to expedite its review by a reader; they should not be construed as limiting the invention in any manner.

### DETAILED DESCRIPTION OF THE INVENTION

The present disclosure relates to methods of treating a subject having post-acute sequelae of COVID-19, abbreviated PASC, and sometimes also referred to under the umbrella term “long COVID.”

In further aspects, the disclosure also relates to methods of preventing or treating the symptoms of PASC. In some further aspects, the disclosure also relates to pharmaceutical combinations used in the disclosed methods, as well as to pharmaceutical kits comprising the disclosed combinations.

While various aspects and features of certain embodiments are summarized above, this detailed description illustrates several exemplary embodiments in further detail to enable one of skill in the art to practice such embodiments, and to make and use the full scope of the invention. However, the described embodiments and examples are provided for illustrative purposes and are not intended to limit the scope of the invention. It will be understood that many modifications, substitutions, changes, and variations in the described embodiments, examples, details, and applications of the invention can be made by those in the art without departing from the spirit of the invention, or the scope of the invention as described in the claims.

### A. GENERAL DEFINITIONS AND TERMS

As used in this specification and the appended claims, the singular forms “a,” “an,” and “the” include plural referents unless the context clearly dictates otherwise. Thus, for example, reference to “an active agent” includes reference to a combination of two or more active agents, and reference to “an excipient” includes reference to a combination of two or more excipients. While the term “one or more” may be used, its absence (or its replacement by the singular) does not signify the singular only, but simply underscores the possibility of multiple agents or ingredients in particular embodiments. The terms “comprising,” “including,” “such as,” and “having” are inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

Unless otherwise indicated, all numbers expressing quantities of ingredients, properties such as concentration, reaction conditions, and so forth, used to describe and claim certain embodiments of the invention are to be understood as being modified in some instances by the term “about.” Accordingly, In embodiments, the numerical parameters set forth in the written description and attached claims are approximations that can vary depending upon the desired properties sought to be obtained by a particular embodiment. In embodiments, “about” refers to plus or minus five percent (±5%) of the recited unit of measure. The term “substantially,” where it is applied to modify a feature or limitation herein, will be read in the context of the invention and in light of the knowledge in the art to provide the appropriate certainty, e.g., by using a standard that is recognized in the art for measuring the meaning of “substantially” as a term of degree, or by ascertaining the scope as would one of skill in the art.

In some embodiments (equivalently as shorthand, “in embodiments”), the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented In embodiments may contain certain errors resulting from the standard deviation found in their respective testing measurements.

Unless defined otherwise, all technical and scientific terms herein have the meaning as commonly understood by one having ordinary skill in the art to which this invention belongs (as shorthand, “one of skill”). Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill) in view of the language used in the claims. The terminology used herein is for the purpose of describing particular embodiments only and is not intended to be limiting.

Generally, the nomenclature used and procedures performed herein are those known in fields relating to one or more aspects of the invention, such as medicine, biology, molecular biology, pharmacology, chemistry, biochemistry, biotechnology, virology, immunology, infectious disease, statistics, and the like, and are those that will be well known and commonly employed in such fields. Standard techniques and procedures will be those generally performed according to conventional methods in the art.

As used herein, the terms “subject,” “user,” “patient,” and “individual” are used interchangeably, and refer to a human, a mammal, or any other animal (e.g., a bird) susceptible to COVID-19 infection. The animal may be a domestic animal (e.g., cats, dogs). The animal may be a livestock or farm animal (e.g., horses, cows, pigs, chickens). Preferably, the subject is a human. The subject may be a human infant, a human child, a human adult, or an elderly human. Such terms will be understood to include one who has an indication for which a method described herein may be efficacious, or who otherwise may benefit by the invention. In general, all of the disclosed methods will be appreciated to work for all individuals, although individual variation is to be expected, and will be understood. The disclosed methods of treatment also can be modified to treat multiple patients at once, including couples or families. Hence, these terms will be understood to also mean two or more individuals.

Herein, “an effective amount,” a “therapeutically effective amount,” or “a pharmacologically effective amount” refers to an amount of an active agent (e.g., an antiviral agent or a monoclonal antibody) that is non-toxic and sufficient to provide the desired therapeutic effect with performance at a reasonable benefit/risk ratio attending any medical treatment. The effective amount will vary depending upon the subject, the weight and age of the subject, the severity of the symptoms or degree of health benefit sought, the manner of administration, and the like, all of which can readily be determined by one of skill in the art.

Herein, “therapeutic effect” or “therapeutic efficacy” means the responses(s) in a subject, and preferably a human, after treatment that are judged to be desirable and beneficial. Hence, depending on the symptoms to be treated, or improvement in health or functioning sought, and depending on the particular constituent(s) of the methods of the disclosure under consideration, those responses shall differ, but would be readily understood by those of skill in the art.

Herein, “treating” or “treatment” of a disorder, such as PASC, includes any treatment of the disorder in a mammal, and preferably in a human, and includes: (a) inhibiting a disorder, i.e., arresting its development; (b) relieving a disorder, i.e., causing regression of the disorder or its symptoms; (c) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder.

In a preferred use, the term “treating” or “treatment” will refer to reducing, eliminating, or alleviating one or more symptoms of PASC in a subject.

In embodiments, treatment will be of a PASC symptom that is the same as any of those of the original SARS-CoV-2 infection, and that persisted following the original SARS-CoV-2 infection.

In embodiments, treatment will be of a PASC symptom that is not the same as any of those of the original SARS-CoV-2 infection. In embodiments, treatment is of a PASC symptom that is different from all of those of the original SARS-CoV-2 infection, and that emerged following the original SARS-CoV-2 infection.

Those of skill in the art will know the appropriate symptoms and corresponding clinically measurable indicators and/or other diagnostic criteria associated with symptoms of PASC. The reduction or alleviation of a symptom includes reducing the incidence or severity of that particular symptom. For example, certain PASC symptoms include fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, and stomach pain. PASC symptoms also commonly include fatigue, headache, sleep problems (e.g., insomnia), dizziness, difficulty thinking or concentration (i.e., brain fog), change or loss in smell or taste, depression, and anxiety. Other such symptoms and clinically measurable indicators, alone or in combination, would be understood to one of skill based on the teachings herein and general knowledge in the art, for example by reference to the “PASCLex” PASC symptom lexicon described in Wang L, et al. J Biomed Inform. 2022 January; 125:103951, incorporated by reference as if fully set forth herein.

As used herein, “COVID” refers to contagious diseases caused by the coronavirus group of RNA viruses and present in a human, a mammal, or any other animal. “COVID-19” refers to the COVID viral contagious disease originating from the coronavirus strain Severe Acute Respiratory Syndrome Coronavirus 2 (SARS-CoV-2) and present in a human, a mammal, or any other animal. COVID-19 also refers to viral diseases originating from a variant of SARS-CoV-2. Variant refers to a change in the genetic code of a virus, wherein the variant has a number of mutations that differentiate it from other variants of a common ancestor virus. Variants of SARS-CoV-2 include, but are not limited to: Alpha, Beta, Gamma, Delta, Omicron, Epsilon, Zeta, Eta, Theta, Iota, Kappa, Labda, Mu, Cluster 5, and any future variants yet to be defined, the ascertainment and appreciation of which shall be within the ordinary skill of those in the art.

Variants of SARS-CoV-2 further include PANGO lineages A.1-A.30, AA.1-AA.8, AB.1, AC.1, AD.1, AD.2, AE.1-AE.8, AF.1, AG.1, AH.1-AH.3, AJ.1, AK.1, AK.2, AL.1, AM.1-AM.4, AN.1, AP.1, AQ.1, AQ.2, AT.1, AS.1, AS.2, AU.1-AU.3, AV.1, AW.1, AY.1-AY.134, AZ.1-AZ.6, B, B.1-B.61, BA.1-BA.5, BB.2, BC.1, BC.2, BD.1, BE.1-BE.13, BF.1-BF.42, BG.1-BG.7, BH.1, BJ.1, BK.1, BL.1-BL.6, BM.1-BM.6, BN.1-BN.6, BP.1, BQ.1, BR.1-BR.5, BS.1, BT.1, BT.2, BU.1-BU.3, BV.1, BV.2, BW.1, BY.1, BZ.1, BZ.2, C.1-C.40, CA.1-CA.7, CB.1, CC.1, CD.1, CD.2, CE.1, CF.1, CG.1, CH.1-CH.3, CJ.1, CK.1-CK.3, CL.1, CM.1-CM.12, CN.1, CN.2, CP.1-CP.8, CQ.1, CQ.2, CR.1, CR.2, CS.1, CT.1, CV.1, CV.2, CW.1, CY.1, CY.2, CZ.1, CZ.2, D.1-D.5, DA.1, DB.1-DB.3, DC.1, DD.1, DE.1, DE.2, DF.1, DG.1, DH.1, DJ.1, DK.1, DL.1, DM.1, DN.1-DN.3, DP.1, DQ.1, DR.1, DR.2, DS.1-DS.3, DT.1-DT.3, DU.1, DV.1-DV.8, DW.1, DY.1-DY.4, DZ.1, DZ.2, EA.1, EA.2, EB.1, EC.1, ED.1-ED.3, EE.1-EE.5, EF.1-EF.3, EG.1-EG.14, EH.1, EJ.1-EJ.3, EK.1-EK.4, EL.1, EM.1, EN.1, EP.1-EP.2, EQ.1, ER.1, ET.1, ES.1, EU.1, EW.1-EW.3, EV.1, EY.1, EZ.1, FA.1, FA.2, FB.1, FB.2, FC.1, FD.1-FD.4, FE.1, FF.1, FG.1-FG.3, FH.1, FJ.1, FK.1, FL.1-FL.36, FM.1, FM.2, FN.1, FP.1-FP.4, FQ.1, FR.1, FR.2, FS.1, FT.1-FT.4, FU.1-FU.5, FV.1, FW.1-FW.3, FY.1-FY.7, FZ.1, FZ.2, G.1, GA.1-GA.6, GB.1, GB.2, GC.1, GC.2, GD.1-GD.3, GE.1, GF.1, GG.1, GH.1, GJ.1-GJ.5, GK.1-GK.4, GL.1, GM.1-GM.3, GN.1-GN.5, GO.1, GP.1-GP.3, GR.1, GS.1-GS.5, GT.1, GU.1, GV.1, GW.1-GW.5, GY.1-GY.8, GZ.1, HA.1, HA.2, HB.1, HC.1-HC.4, HD.1, HE.1, HF.1, HG.1, HG.2, HH.1, HH.2, HJ.1, HK.1-HK.9, HK.11, HL.1, HL.2, HM.1, HN.1, HP.1, HQ.1, HR.1, HS.1, HT.1, HT.2, HU.1, HU.2, HV.1, HW.1, HY.1, HZ.1-HZ.3, JA.1, JB.1, JB.2, JC.1, JD.1, JD.2, JE.1, JF.1, JF.2, JG.1-JG.3, JH.1, JH.2, JJ.1, JK.1, JL.1, JM.1, JM.2, JN.1, JP.1, K.1-K.3, L.1-L.4, M.1-M.3, N.1-N. 10, P.1-P.7, Q.1-Q.8, R.1, R.2, S.1, XA, XB, XC, XD, XE, XF, XG, XH, XJ, XK, XL, XM, XN, XP, XQ, XR, XS, XT, XU, XV, XW, XY, XZ, XAA, XAB, XAC, XAD, XAE, XAF, XAG, XAH, XAJ, XAK, XAL, XAM, XAN, XAP, XAQ, XAR, XAS, XAT, XAU, XAV, XAW, XAY, XAY.1-XAY.3, XAZ, XBA, XBB, XBB.1-XBB.9, XBC, XBC.1, XBC.2, XBD, XBE, XBF, XBF.1-XBF.10, XBG, XBH, XBJ, XBJ.1-XBJ.4, XBK, XBK.1, XBL, XBL.1-XBL.3, XBM, XBN, XBP, XBQ, XBR, XBW, XBY, XBZ, XCA, XCB, XCC, XCD, XCE, XCF, XCF.1, XCF.2, XCG, XCH, XCH.1, XCJ, XCK, XCL, XCM, XCN, XCP, U.1-U.3, V.1, V.2, W.1-W.4, Y.1, Z.1, respective sub-lineages and any future lineages yet to be defined. Variants of SARS-CoV-2 further include, GISAID clades S, L, O, V, G, GH, GR, GV, and any future clades yet to be defined, the ascertainment and appreciation of which shall be within the ordinary skill of those in the art. Variants of SARS-CoV-2 further includes Nextstrain clades 19A.19B, 20A, 20B, 20C, 20D, 20E, 20F, 20G, 20H, 20I, 20J, and any future clades yet to be defined, the ascertainment and appreciation of which shall be within the ordinary skill of those in the art.

Herein, “post acute sequelae of COVID-19” and “PASC,” used interchangeably with the terms “long COVID” and “long COVID-19,” refer to the continuance, occurrence, or emergence of COVID-19 symptoms past the standard incubation period, transmission period, or symptomatic period of a COVID infection, as such periods will be known to those in the art. To delimit a COVID-19 syndrome or symptom from such sequela(e) or from PASC, where not otherwise defined or suggested herein, such sequela(e) shall present more than 28 days after the first positive PCR test in a patient that proved a SARS-CoV-2 infection, or otherwise from the date selected as the date of infection, as would be understood to those in the art, or shall persist for longer than 28 days after the date of infection (see also Thaweethai T, et al. JAMA. 2023; 329(22):1934-1946). In embodiments, sequela(e) shall persist for 30 or more days after infection. In embodiments, sequela(e) shall persist for four weeks or more after infection. In embodiments, sequela(e) shall persist for about four weeks or more after infection. In embodiments, sequela(e) shall persist for over 1 month after infection, including for over 2 months, over 3 months, over four months, over 5 months, over 6 months, and longer, including over 1 year. In embodiments, sequela(e) shall persist for over 3 months after infection, and last for at least 2 months. In embodiments, sequela(e) shall present 30 or more days after infection. In embodiments, sequela(e) shall present four weeks or more after infection. In embodiments, sequela(e) shall present about four weeks or more after infection. In embodiments, sequela(e) shall present over 1 month after infection, including over 2 months, over 3 months, over four months, over 5 months, over 6 months, and longer, including over 1 year after infection. In embodiments, sequela(e) shall present over 3 months after infection, and last for at least 2 months. Other definitions that may be used for the emergence and/or persistence of sequelae will be known to those of skill, and include (quoting Sherif Z A, et al. Elife. 2023; 12: e86002): “the continuation of COVID-19 symptoms—or the emergence of new ones—4 or more weeks after infection with SARS-CoV-2 (http://www.CDC.gov), or those persistently symptomatic for >30 days from onset of infection (http://www.NIH.gov)” or symptoms occurring “usually 3 months from the onset of COVID-19 . . . that last for at least 2 months and cannot be explained by an alternative diagnosis (http://www.WHO.int),” or “ongoing, relapsing, or new symptoms, or other health effects occurring after the acute phase of SARS-CoV-2 infection (i.e., present four or more weeks after the acute infection).”

Where not otherwise defined or known by context, the terms “sequela” or “sequelae,” or broadly the term “PASC” and its synonyms herein, will refer to any conditions(s), disease(s), syndrome(s), symptom(s), or disability(ies) that become manifest in a patient following a SARS-CoV-2 infection, or that persist for a period (such as above) after an initial SARS-CoV-2 infection, and that are most probably causally related to the SARS-CoV-2 infection, regardless of whether the patient developed any syndromes or symptoms of COVID-19 or stayed free of them. In embodiments, such sequelae are different from the known spectrum of syndromes and symptoms of COVID-19. In embodiments, sequelae are different from the syndromes and symptoms of the initial SARS-CoV-2 infection reported by the subject. That is, in some embodiments herein, disclosed compositions and methods are used to treat sequelae as are different from the syndromes and symptoms of the initial SARS-CoV-2 infection reported by a subject (i.e., “new” symptoms). In embodiments, sequelae are the same as the syndromes and symptoms of the initial SARS-CoV-2 infection reported by the subject, but are reported as persisting for a period (such as above) after the initial SARS-CoV-2 infection.

Post acute “sequelae” refer to, as examples, fatigue (including chronic fatigue syndrome or CFS), shortness of breath (dyspnea), (long-lasting) cough, joint pain, chest pain and muscle weakness, difficulties with thinking and concentration (“brain fog”), depression, muscle pain, headache, intermittent fever, fast beating (or pounding) heart (“heart palpitations”), inflammation of the heart muscle, lung function abnormalities, acute kidney injury, rash, hair loss, teeth loss, sleep disorders, insomnia, memory problems, anxiety, mood swings, low-grade fever, needle pains in arms and legs, diarrhea, bouts of vomiting, sore throat, swallowing difficulties, and new onset of diabetes and hypertension. In embodiments, post acute sequelae will include in particular neurologic or psychiatric sequelae, further including any of (ischemic) stroke, bilateral frontotemporal hypoperfusion, intracranial hemorrhage, paralysis, hemiplegia, ataxia, cranial nerve deficits, encephalopathy, acute necrotizing encephalitis, delirium, meningitis, seizures, obsessive-compulsive disorders, Parkinson's disease, Alzheimer's disease, accelerated aging, aphasia, cognitive decline, reduced level of consciousness, and Guillain-Barre syndrome.

In embodiments, post acute sequelae include any of anorexia, anxiety, arrhythmias, confusion (“brain fog”), dementia, depression, dyspnea, fatigue, hair loss, headache, heart failure, cardiomyopathy, angina, hepatic dysfunction, hyperglycemia, type 2 diabetes, increased heart rate, inflammation, loss of appetite, loss of memory, loss of smell, mood disorder, muscle weakness, myocardial ischemia, post-exertional malaise, diminished neurocognition, diminished sensory function, pulmonary infiltrates or fibrosis, postural orthostatic hypotension, renal dysfunction, and respiratory distress. In embodiments, sequelae include any of myalgia, fibromyalgia, idiopathic pulmonary fibrosis, fatigue (including persistent fatigue, exertional fatigue, and muscle fatigue), mitochondrial dysfunction, dyspnea after exertion, postural orthostatic tachycardia syndrome, tachycardia, mood disorders, and depression.

Other post acute sequelae, which herein also may be referred to as a “symptom of PASC” or a “symptom of post-acute sequelae of COVID-19” will be known to those of skill by reference to the disclosure herein, including any of the definitions herein, in combination with the general knowledge in the art.

Herein, “antiviral agent” refers to agents intended for the use of combating a viral infection in a human, a mammal, or any other animal. For example, antiviral agents may include: uncoating inhibitors, entry inhibitors, protease inhibitors, immunostimulatory agents, latency reversal agents, reverse transcriptases, reverse transcriptase inhibitors, integrate inhibitors, nucleoside analogues, replication inhibitors, polymerase inhibitors (e.g., RNA polymerase inhibitors), agents that act on viral uncapping, neuraminidase inhibitors, fusion inhibitors, interferons, nucleoside reverse transcriptase inhibitors (NRTIs), and non-nucleoside reverse transcriptase inhibitors.

Herein, “interferon” refers to a singular or multiple of signaling proteins. For example, the interferons may be produced by white cells, fibroblasts, lymphocytes or other host cells in response to viral presence. Interferons may be naturally derived or manufactured. “Interferon” further refers to interferons classified as alpha, beta, gamma, and relevant subclasses. Additionally, “interferon” refers to type I, type II, and type III interferons and relevant subtypes.

Herein, “monoclonal antibody” refers to an antibody originating from a singular cell line or single clone. For example, monoclonal antibodies include: whole monoclonal, fragment antigen-binding, single-chain variable fragment, single domain antibody, trifunctional, chemically linked, and bi-specific T-cell engager antibodies. “Monoclonal antibody” further refers to any of an IgA, IgD, IgE, IgG, or IgM antibody, an anti-B lymphocyte stimulator (anti-BlyS) antibody (e.g., belimumab, BENLYSTA®), a humanized antibody, a synthetic antibody, a long-acting monoclonal antibody, a broadly-neutralizing monoclonal antibody, and any derivative thereof. “Long-acting monoclonal antibody” refers to an antibody that has a longer half-life relative to a standard monoclonal antibody. “Broadly-neutralizing monoclonal antibody” refers to an antibody that activates other immune cells in addition to a standard monoclonal antibody target. Monoclonal antibodies may target any existing SARS-CoV-2 variant or future variants or lineages yet to be defined.

Herein, “polyclonal antibody” refers to a heterogeneous mixture of antibodies. For example, a polyclonal antibody may be derived from multiple cell lines or multiple clones. A polyclonal antibody may be a combination of, but not limited to any of an IgA, IgD, IgE, IgG, or IgM antibody, an anti-BlyS antibody, a humanized antibody, a synthetic antibody, a long-acting antibody, a broadly-neutralizing antibody, and any derivative thereof. “Long-acting polyclonal antibody” refers to an antibody that has a longer half-life relative to a standard polyclonal antibody. “Broadly-neutralizing polyclonal antibody” refers to an antibody that activates other immune cells in addition to a standard monoclonal antibody target. Polyclonal antibodies may target any existing SARS-CoV-2 variant or future variants or lineages yet to be defined.

### B. PHARMACEUTICAL AGENTS

In some aspects are disclosed pharmaceutical combinations of two or more pharmaceutical agents, which may be antiviral agents, antibodies, or additional active agents, such as described herein and below.

a. Antiviral Agents

In some aspects are disclosed pharmaceutical combinations of two or more pharmaceutical agents, wherein at least one pharmaceutical agent is an antiviral agent.

In embodiments, the antiviral agent is a protease inhibitor.

In embodiments, the antiviral agent is an inhibitor of SARS-CoV-2 main protease (“Mpro inhibitor”) (see Huang C, et al. Signal Transduct Target Ther. 2023 Mar. 16; 8(1):128). In embodiments, the Mpro inhibitor is nirmatrelvir, PAXLOVID®, ensitrelvir (e.g., XOCOVA®), ensitrelvir, simnotrelvir, simnotrelvir and ritonavir (e.g., as XIANNUOXIN®), or SY110 (see Huang, op cit.).

In embodiments, the antiviral agent is an inhibitor of SARS-CoV-2 RNA-dependent RNA polymerase (“RdRp inhibitor”). In embodiments, the RdRp inhibitor is remdesivir, molnupiravir, renmindevir, or azvudine.

In embodiments, the antiviral agent is an ACE2 decoy receptor drug. Without being bound by theory, such drugs are soluble molecules having a structure similar to the typically membrane-bound ACE2 receptor, and which are able to recognize and bind to the spike protein of SARS-Cov-2 variants, neutralizing the virus before it can infect a host cell (Torchia J A, et al. Sci Adv. 2022 Dec. 9; 8(49): eabq6527).

In embodiments, the antiviral agent is any of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, and telaprevir.

In embodiments, the antiviral agent is any of molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), and acyclovir.

In some embodiments where an antiviral agent is sofosbuvir, sofosbuvir is administered together with daclatasvir. In some embodiments, sofosbuvir is administered together with daclatisvir and ribavirin. In embodiments, sofosbuvir is administered together with ledipasvir (e.g., as HARVONI™). In embodiments, the antiviral agent is any of sofosbuvir, daclatasvir, ribavarin, or ledipsavir, including all combinations thereof.

In embodiments, an antiviral agent may be provided as a pharmaceutically acceptable salt. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, I-aspartate, I-camsylate, I-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (Berge et al. (1977) J. Pharm. Sci. 66:1-19.)

b. Antibodies

In some aspects are disclosed methods of using pharmaceutical combinations of two or more pharmaceutical agents, wherein at least one pharmaceutical agent is an antibody.

In embodiments, the antibody is any of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casirivirimab, imdevimab, etesevimab, bamlanivimab, amubarvimab, regdanvimab, romlusevimab, adintrevimab, 002-S21F2, and durvalumab.

In embodiments, the antibody is any of AZD3152, Lenzilumab, Ravulizumab, Canakinumab, Adalimumab, Sarilumab, Ronapreve, Vilobelimab, Casirivimab, Sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, and AER002.

In embodiments, the antibody is a monoclonal antibody.

In embodiments, the antibody is a polyclonal antibody.

In embodiments, the antibody is an Immunoglobulin A (IgA) antibody, an Immunoglobulin D (IgD) antibody, an Immunoglobulin E (IgE) antibody, an Immunoglobulin G (IgG) antibody, or an Immunoglobulin M (IgM) antibody. In some preferred embodiments, the antibody is an IgG antibody.

In embodiments, the antibody is an IgG1 or IgG3 antibody (i.e., an IgG antibody in subclasses IgG1 or IgG3). In embodiments, the antibody is an IgG2 or IgG4 antibody.

In embodiments, the antibody is an anti-BlyS antibody.

In embodiments, the antibody is a broadly-neutralizing antibody.

In embodiments, the antibody is a broadly-neutralizing monoclonal antibody.

In embodiments, the antibody is a long-acting antibody.

In embodiments, the antibody is a long-acting monoclonal antibody.

In embodiments, the antibody is an anti-spike antibody, as would be understood to those of skill (see, e.g., Mikulska M, et al. Clin Infect Dis. 2023 Jul. 26; 77(2):280-286).

In embodiments, the antibody is selected to remain bioavailable for a longer period of time in a subject. In embodiments, the antibody is an IgG antibody selected to remain bioavailable longer in a subject.

“Longer,” in embodiments, may refer to a half-life of over one month, to over one year. In embodiments, “longer” refers to a period of time or a relative proportion, versus a comparator, the selection of which, and the calculation of the comparison, will be known to those of skill. For instance, longer may refer to a period of over one month longer than a comparator, or may refer to a bioavailability of greater than 10%, 20%, 50%, or 100%, as example, more than a comparator. In some preferred embodiments, where an antibody remains bioavailable longer in a subject, the antibody will remain clinically active during the period.

In embodiments, an antibody remains longer in a subject because of a synergistic effect with another pharmaceutical agent in the pharmaceutical combination, including an antiviral agent, another antibody, or an additional active agent. In embodiments, an additional active agent is selected for its synergy with an antibody, where such synergy increases the time period of bioactivity of the antibody in a subject.

In embodiments, an antibody is specifically engineered to remain bioavailable for a longer period of time in a subject. In embodiments, an antibody is an IgG antibody specifically engineered to remain bioavailable longer in a subject. Various methods will be known to those in the art for antibody engineering and which can be used for mAbs to add their SARS-CoV-2 binding specificity to IgG scaffolds that remain bioavailable longer in a subject. In addition to factors related to antigen specificity, such as binding affinity and avidity, the mechanism of action (MoA) of therapeutic monoclonal antibodies (mAbs) encompasses Fc-mediated effector functions. These functions include Antibody-Dependent Cellular Cytotoxicity (ADCC) and its closely associated Antibody-Dependent Cellular Phagocytosis (ADCP). To evaluate these effector functions, various cell-based assays can be used to examine different combinations of effector and target cells, with multiple readout methods like Fcγ receptor (FcγR)-mediated lysis, fluorescence, or luminescence. To optimize FcγR-mediated effector functions for clinical safety and efficacy, several modification strategies can be employed. Such strategies will be understood to involve techniques such as introducing point mutations, altering glycosylation patterns, combining different Fc subclasses (cross-isotypes), and truncating the Fc portion of the mAb. Such techniques can permit the development of additional therapeutic mAbs, which will be understood to be useful in the invention. In embodiments, the FcγR-mediated effector functions will be considered when assessing the effectiveness of therapeutic monoclonal antibodies, based on their role in the MoA. In embodiments, consideration will be of the ADCC, ADCP, or other aspects of the MoA.

In embodiments, an antibody is selected based on its neutralizing activity against the circulating strains at the time of the selection. In embodiments, an antibody is selected based on its neutralizing activity against the strains circulating prior to the time of the selection, such as did or had a high likelihood of causing PASC or a PASC symptom in a subject to be administered the antibody. In embodiments, an antibody is selected both for its neutralizing activity against circulating strains and against prior strains.

In embodiments, an antibody has reduced Fc receptor binding. In embodiments, an antibody is selected based on its level of Fc receptor binding, such as for having reduced Fc receptor binding relative to a comparator. In some embodiments, wherein an antibody has reduced Fc receptor binding, the antibody will have a reduced risk of antibody-dependent enhancement of viral infection or enhancement of disease.

In embodiments, an antibody has reduced complement C1q binding. In embodiments, an antibody is selected based on its complement C1q binding, such as for having reduced complement C1q binding relative to a comparator.

c. Additional Active Agents

In some aspects are disclosed methods of using pharmaceutical combinations of two or more pharmaceutical agents, and further comprising an additional active agent.

In embodiments, the additional active agent is selected to provide an additional therapeutic effect, such as antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, empathogenic, entactogenic, entheogenic, psychedelic, dissociative, nootropic, euphoric, sedative, and stimulant effects. In embodiments, the additional active agent may be any of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, monoamine oxidase inhibitors, empathogens, entactogens, entheogens, psychedelics, dissociatives, nootropics, euphorics, sedatives, stimulants, and vitamins. These active agents may be in ion, freebase, or salt form, include polymorphs, and may be isomers.

In embodiments, the additional active agent is any of an uncoating inhibitor, entry inhibitor, protease inhibitor, immunostimulatory agent, latency reversal agent, reverse transcriptase, reverse transcriptase inhibitor, integrate inhibitor, nucleoside analogue, RNA polymerase inhibitor, viral uncapping agent, neuraminidase inhibitor, fusion inhibitor, interferon, nucleoside reverse transcriptase inhibitor, or non-nucleoside reverse transcriptase inhibitor.

In embodiments, the additional active agent is an immunostimulatory agent, an antiviral agent, or an antibody. In embodiments, the additional active agent is an immunostimulatory agent. Without being bound by theory, immunostimulatory agents are agents that stimulate the immune system. When used in a disclosed method or combination, an immunostimulatory agent may provide therapeutic or synergistic benefits, resulting in an increase in the treatment efficacy of a disclosed method or combination. Exemplary immunostimulatory agents include latency reversal agents, cationic peptides, cationic polypeptides, protamine, protamine, nucleolin, spermine, spermidine, cationic polysaccharides, chitosan, TDM, MDP, muramyl dipeptide, pluronic, alum solution, aluminum hydroxide, and polyphosphazene. In embodiments, the additional active agent is an antibody, such as a monoclonal antibody, a polyclonal antibody, a broadly-neutralizing monoclonal antibody, a long-acting monoclonal antibody, a broadly-neutralizing polyclonal antibody, or a long-acting polyclonal antibody.

In embodiments, the additional active agent is a COVID-19 vaccine. Exemplary COVID-19 vaccines include COMIRNATY® (Pfizer-BioNTech), SPIKEVAX® (Moderna), COVAXIN® (Bharat Biotech), NVX-CoV2373 (Novavax), and others as will be readily appreciated by those in the art.

In embodiments, the additional active agent is an additional agent having antiviral activity. When used in a disclosed method or combination, an additional agent having antiviral activity may provide therapeutic or synergistic benefits, resulting in an increase in the treatment efficacy of a disclosed method or combination. Exemplary additional agents having antiviral activity include uncoating inhibitors, entry inhibitors, protease inhibitors, immunostimulatory agents, latency reversal agents, reverse transcriptases, reverse transcriptase inhibitors, integrate inhibitors, nucleoside analogues, RNA polymerase inhibitors, agents that act on viral uncapping, neuraminidase inhibitors, fusion inhibitors, interferons, nucleoside reverse transcriptase inhibitors (NRTIs), and non-nucleoside reverse transcriptase inhibitors.

In embodiments, the additional agent having antiviral activity is any of MIR 19® (siR-7-EM/KK-46), baicalein, peginterferon lambda, metformin, ursodeoxycholic acid (UCDA), TVGN-489, LAU-7b, sabizabulin, azithromycin, ivermectin, opanganib, baricitinib, mycophenolate, APN01, epigallocatechin gallate (EGCG), diphenhydramine, quercetin, a Chinese herbal formulation, nicotine, lactoferrin, nattokinase, or ubiquinone.

In embodiments, the Chinese herbal formulation may be any of Qing-Fei-Pai-Du-Tang (QFPDT), Lianhua Qingwen (LQ), Taiwan Chingguan Yihau (NRICM101), Jing Si herbal drink (JSHD).

In embodiments, the additional agent having antiviral activity is a viral uncoating inhibitor. In embodiments, the additional agent having antiviral activity is a viral entry inhibitor. In embodiments, the additional agent having antiviral activity is a viral replication inhibitor. In embodiments, the additional agent having antiviral activity is a viral polymerase inhibitor. In embodiments, the additional agent having antiviral activity is a polyclonal antibody. In embodiments, the additional agent having antiviral activity is a broadly-neutralizing monoclonal antibody. In embodiments, the additional agent having antiviral activity is a long-acting monoclonal antibody.

In embodiments, the additional agent having antiviral activity is an interferon. In embodiments, the interferon is an alpha interferon, beta interferon, gamma interferon, or any combination thereof. In embodiments, the interferon is an alpha interferon. In embodiments the interferon is a beta interferon. In embodiments the interferon is a gamma interferon. In embodiments the interferon is a type I interferon, type II interferon, type III interferon, or any combination thereof. In embodiments the interferon is a type I interferon. In embodiments the interferon is a type II interferon. In embodiments the interferon is a type III interferon. In embodiments, the interferon is selected from the group consisting of interferon alfa-n3, interferon alfa-2b, interferon alfacon-1, peginterferon alfa-2a, interferon beta-1a, interferon beta-1b, and interferon gamma1-b.

In embodiments, the additional agent having antiviral activity is another compound having such activity as known to those in the art, for example as described in Banerjee S, et al. Vaccines (Basel). 2023 Feb. 1; 11(2):332, the complete disclosure of which is incorporated by reference as if fully set forth herein.

In embodiments, the additional agent having antiviral activity is any antiviral agent as described and listed in the section on Antiviral Agents above. In embodiments, for example, a pharmaceutical combination may comprise a first antiviral agent, a second antiviral agent, and an additional active agent, which is a third antiviral agent, selected from the same lists.

In embodiments, an additional active agent may be provided as a pharmaceutically acceptable salt.

In embodiments, an additional active agent acts to increase a therapeutic effect, provide an additional therapeutic effect, decrease an unwanted effect, increase stability and/or shelf-life, improve bio-availability, induce synergy or provide synergistic effects, or alter pharmacokinetics or pharmacodynamics.

In embodiments, the additional active agent provides synergistic effects, or is selected to provide synergistic effects. In embodiments, “synergistic effects” will be understood to include increases in potency, bioactivity, bioaccessibility, bioavailability, or therapeutic effect, that are greater than the additive contributions of the components acting alone, and/or are greater than the contribution of the isolated compounds on their own. Numerous methods known to those of skill in the art exist to determine whether there is synergy as to a particular effect, i.e., whether, when two or more components are mixed together, the effect is greater than the sum of the effects of the individual components when applied alone, thereby producing “1+1>2.” Suitable methods include the isobologram analysis (or contour method) (Huang, Front Pharmacol., 2019; 10:1222) and the equation of Loewe additivity (Loewe & Muischnek, 1926, Arch. Exp. Pathol Pharmacol. 114:313-326). A synergistic effect also may be calculated using methods such as the Sigmoid-Emax equation (Holford & Scheiner, 1981, Clin. Pharmacokinet. 6:429-453) and the median-effect equation (Chou & Talalay, 1984, Adv. Enzyme Regul. 22:27-55). The corresponding graphs associated with the equations referred to above are the concentration-effect curve and combination index curve, respectively. Each equation may be applied to experimental data to generate a corresponding graph to aid in assessing the effects of the drug combination.

### C. PHARMACEUTICAL COMBINATIONS

In some aspects are disclosed pharmaceutical combinations comprising two or more disclosed pharmaceutical agents, and which may be used in the disclosed methods. In embodiments, a “pharmaceutical combination” refers to a combination of disclosed pharmaceutical agents, which may be used in the disclosed methods, and which comprises two or more of: i) a first antiviral agent, or a pharmaceutically acceptable salt thereof; ii) a second antiviral agent, or a pharmaceutically acceptable salt thereof; iii) a first antibody; iv) a second antibody; and v) an additional active agent.

a. First Antiviral and First Antibody

In embodiments, a pharmaceutical combination comprises: i) a first antiviral agent, or a pharmaceutically acceptable salt thereof; and ii) a first antibody.

In embodiments, the first antiviral agent is a protease inhibitor. In embodiments, the first antiviral agent is an Mpro inhibitor. In embodiments, the first antiviral agent is an RdRp inhibitor. In embodiments, the first antiviral agent is an ACE2 decoy receptor drug. In embodiments, the first antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, and telaprevir.

In embodiments, the first antiviral agent is selected from the group consisting of molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), and acyclovir.

In embodiments, the first antiviral agent is ritonavir. In embodiments, the first antiviral agent is nirmatrelvir. In embodiments, the first antiviral agent is remdesivir. In embodiments, the first antiviral agent is amprenavir. In embodiments, the first antiviral agent is remdesivir. In embodiments, the first antiviral agent is atazanavir. In embodiments, the first antiviral agent is darunavir. In embodiments, the first antiviral agent is fosamprenavir. In embodiments, the first antiviral agent is indinavir. In embodiments, the first antiviral agent is lopinavir. In embodiments, the first antiviral agent is nelfinavir. In embodiments, the first antiviral agent is saquinavir. In embodiments, the first antiviral agent is tipranavir. In embodiments, the first antiviral agent is asunaprevir. In embodiments, the first antiviral agent is boceprevir. In embodiments, the first antiviral agent is grazoprevir. In embodiments, the first antiviral agent is glecaprevir. In embodiments, the first antiviral agent is paritaprevir. In embodiments, the first antiviral agent is simeprevir. In embodiments, the first antiviral agent is telaprevir. In embodiments, the first antiviral agent is molnupiravir. In embodiments, the first antiviral agent is GC376. In embodiments, the first antiviral agent is arbidol (umifenovir). In embodiments, the first antiviral agent is SSAA09E2. In embodiments, the first antiviral agent is ribavirin. In embodiments, the first antiviral agent is favilavir. In embodiments, the first antiviral agent is oseltamivir. In embodiments, the first antiviral agent is zanamivir. In embodiments, the first antiviral agent is abacavir. In embodiments, the first antiviral agent is stavudine. In embodiments, the first antiviral agent is valganciclovir. In embodiments, the first antiviral agent is ganciclovir. In embodiments, the first antiviral agent is cidofovir. In embodiments, the first antiviral agent is entecavir. In embodiments, the first antiviral agent is lamivudine. In embodiments, the first antiviral agent is maraviroc. In embodiments, the first antiviral agent is azidothymidine. In embodiments, the first antiviral agent is nelfinavir. In embodiments, the first antiviral agent is dolutegravir. In embodiments, the first antiviral agent is olsetamivir. In embodiments, the first antiviral agent is ensitrelvir (Xocova). In embodiments, the first antiviral agent is simnotrelvir. In embodiments, the first antiviral agent is bemnifosbuvir (AT-527). In embodiments, the first antiviral agent is deuremidevir (VV116). In embodiments, the first antiviral agent is azvudine. In embodiments, the first antiviral agent is sofosbuvir. In embodiments, the first antiviral agent is obeldesivir (GS-5245). In embodiments, the first antiviral agent is acyclovir.

In embodiments, the first antiviral agent is obeldesivir.

In embodiments, the first antiviral agent is ensitrelvir.

In embodiments, the first antiviral agent is not a protease inhibitor. In embodiments, the first antiviral agent is not an Mpro inhibitor. In embodiments, the first antiviral agent is not an RdRp inhibitor. In embodiments, the first antiviral agent is not an ACE2 decoy receptor drug.

In embodiments, the first antiviral agent is not ritonavir. In embodiments, the first antiviral agent is not nirmatrelvir. In embodiments, the first antiviral agent is not remdesivir. In embodiments, the first antiviral agent is not amprenavir. In embodiments, the first antiviral agent is not remdesivir. In embodiments, the first antiviral agent is not atazanavir. In embodiments, the first antiviral agent is not darunavir. In embodiments, the first antiviral agent is not fosamprenavir. In embodiments, the first antiviral agent is not indinavir. In embodiments, the first antiviral agent is not lopinavir. In embodiments, the first antiviral agent is not nelfinavir. In embodiments, the first antiviral agent is not saquinavir. In embodiments, the first antiviral agent is not tipranavir. In embodiments, the first antiviral agent is not asunaprevir. In embodiments, the first antiviral agent is not boceprevir. In embodiments, the first antiviral agent is not grazoprevir. In embodiments, the first antiviral agent is not glecaprevir. In embodiments, the first antiviral agent is not paritaprevir. In embodiments, the first antiviral agent is not simeprevir. In embodiments, the first antiviral agent is not telaprevir. In embodiments, the first antiviral agent is not molnupiravir. In embodiments, the first antiviral agent is not GC376. In embodiments, the first antiviral agent is not arbidol (umifenovir). In embodiments, the first antiviral agent is not SSAA09E2. In embodiments, the first antiviral agent is not ribavirin. In embodiments, the first antiviral agent is not favilavir. In embodiments, the first antiviral agent is not oseltamivir. In embodiments, the first antiviral agent is not zanamivir. In embodiments, the first antiviral agent is not abacavir. In embodiments, the first antiviral agent is not stavudine. In embodiments, the first antiviral agent is not valganciclovir. In embodiments, the first antiviral agent is not ganciclovir. In embodiments, the first antiviral agent is not cidofovir. In embodiments, the first antiviral agent is not entecavir. In embodiments, the first antiviral agent is not lamivudine. In embodiments, the first antiviral agent is not maraviroc. In embodiments, the first antiviral agent is not azidothymidine. In embodiments, the first antiviral agent is not nelfinavir. In embodiments, the first antiviral agent is not dolutegravir. In embodiments, the first antiviral agent is not olsetamivir. In embodiments, the first antiviral agent is not ensitrelvir (Xocova). In embodiments, the first antiviral agent is not simnotrelvir. In embodiments, the first antiviral agent is not bemnifosbuvir (AT-527). In embodiments, the first antiviral agent is not deuremidevir (VV116). In embodiments, the first antiviral agent is not azvudine. In embodiments, the first antiviral agent is not sofosbuvir. In embodiments, the first antiviral agent is not obeldesivir (GS-5245). In embodiments, the first antiviral agent is not acyclovir.

In embodiments, the first antiviral agent is an antiviral agent from any of the lists of “additional agents having antiviral activity” in the section on additional active agents.

In embodiments, the first antiviral agent is ritonavir or nirmatrelvir. In some embodiments, the first antiviral agent is ritonavir. In other embodiments, the first antiviral agent is nirmatrelvir.

In embodiments, the first antibody (herein as shorthand, “first Ab”; and “Ab” generally used equivalently herein, and as shorthand, for “antibody”) is a monoclonal antibody. In embodiments, the first Ab is a polyclonal antibody. In embodiments, the first Ab is an IgA antibody. In embodiments, the first Ab is an IgD antibody. In embodiments, the first Ab is an IgE antibody. In embodiments, the first Ab is an IgG antibody. In embodiments, the first Ab is an IgM antibody. In embodiments, the first Ab is an anti-BlyS antibody. In some preferred embodiments, the first Ab is an IgG antibody. In embodiments, the first Ab is a broadly-neutralizing antibody. In embodiments, the first Ab is a broadly-neutralizing monoclonal antibody. In embodiments, the first Ab is a long-acting antibody. In embodiments, the first Ab is a long-acting monoclonal antibody. In embodiments, the first Ab is an anti-spike antibody. In embodiments, the first Ab is an IgG1 or IgG3 antibody. In embodiments, the first Ab is an IgG2 or IgG4 antibody.

In embodiments, the first antibody is not an IgA antibody. In embodiments, the first Ab is not an IgD antibody. In embodiments, the first Ab is not an IgE antibody. In embodiments, the first Ab is not an IgG antibody. In embodiments, the first Ab is not an IgM antibody. In embodiments, the first Ab is not an anti-BlyS antibody. In embodiments, the first Ab is not a broadly-neutralizing antibody. In embodiments, the first Ab is not a broadly-neutralizing monoclonal antibody. In embodiments, the first Ab is not a long-acting antibody. In embodiments, the first Ab is not a long-acting monoclonal antibody. In embodiments, the first Ab is not an anti-spike antibody. In embodiments, the first Ab is not an IgG1 or IgG3 antibody. In embodiments, the first Ab is not an IgG2 or IgG4 antibody.

In embodiments, the first antibody is selected from the group consisting of (equivalent to “any of”): tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casirivirimab, imdevimab, etesevimab, bamlanivimab, amubarvimab, regdanvimab, romlusevimab, adintrevimab, 002-S21F2, and durvalumab.

In embodiments, the first antibody is any of AZD3152, Lenzilumab, Ravulizumab, Canakinumab, Adalimumab, Sarilumab, Ronapreve, Vilobelimab, Casirivimab, Sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, and Anti-Endothelin Receptor B Antibody (AER002).

In embodiments, the first antibody is tixagevimab. In embodiments, the first Ab is cilgavimab. In embodiments, the first Ab is pembrolizumab. In embodiments, the first Ab is nivolumab. In embodiments, the first Ab is bevacizumab. In embodiments, the first Ab is ocrelizumab. In embodiments, the first Ab is rituximab. In embodiments, the first Ab is daratumumab. In embodiments, the first Ab is pertuzumab. In embodiments, the first Ab is trastuzumab. In embodiments, the first Ab is infliximab. In embodiments, the first Ab is tocilizumab. In embodiments, the first Ab is atezolizumab. In embodiments, the first Ab is tositumomab. In embodiments, the first Ab is olaratumab. In embodiments, the first Ab is rituximab. In embodiments, the first Ab is basiliximab. In embodiments, the first Ab is ibritumomab tiuxetan. In embodiments, the first Ab is cetuximab. In embodiments, the first Ab is natalizumab. In embodiments, the first Ab is panitumumab. In embodiments, the first Ab is ranibizumab. In embodiments, the first Ab is eculizumab. In embodiments, the first Ab is ofatumumab. In embodiments, the first Ab is belimumab. In embodiments, the first Ab is ipilimumab. In embodiments, the first Ab is pertuzumab. In embodiments, the first Ab is raxibacumab. In embodiments, the first Ab is obinutuzumab. In embodiments, the first Ab is siltuximab. In embodiments, the first Ab is ramucirumab. In embodiments, the first Ab is vedolizumab. In embodiments, the first Ab is alemtuzumab. In embodiments, the first Ab is necitumumab. In embodiments, the first Ab is dinutuximab. In embodiments, the first Ab is elotuzumab. In embodiments, the first Ab is reslizumab. In embodiments, the first Ab is bezlotoxumab. In embodiments, the first Ab is obiltoxaximab. In embodiments, the first Ab is avelumab. In embodiments, the first Ab is sotrovimab. In embodiments, the first Ab is bebtelovimab. In embodiments, the first Ab is casirivirimab. In embodiments, the first Ab is imdevimab. In embodiments, the first Ab is etesevimab. In embodiments, the first Ab is bamlanivimab. In embodiments, the first Ab is amubarvimab. In embodiments, the first Ab isregdanvimab. In embodiments, the first Ab is romlusevimab. In embodiments, the first Ab is adintrevimab. In embodiments, the first Ab is 002-S21F2. In embodiments, the first Ab is AZD3152. In embodiments, the first Ab is lenzilumab. In embodiments, the first Ab is ravulizumab. In embodiments, the first Ab is canakinumab. In embodiments, the first Ab is adalimumab. In embodiments, the first Ab is sarilumab. In embodiments, the first Ab is ronapreve. In embodiments, the first Ab is vilobelimab. In embodiments, the first Ab is casirivimab. In embodiments, the first Ab is sarilumab. In embodiments, the first Ab is leronlimab. In embodiments, the first Ab is golimumab. In embodiments, the first Ab is tabalumab. In embodiments, the first Ab is veltuzumab. In embodiments, the first Ab is mepolizumab. In embodiments, the first Ab is secukinumab. In embodiments, the first Ab is evolocumab. In embodiments, the first Ab is blinatumomab. In embodiments, the first Ab is adotrastuzumab. In embodiments, the first Ab is brentuximab. In embodiments, the first Ab is ipilumumab. In embodiments, the first Ab is denosumab. In embodiments, the first Ab is ustekinumab. In embodiments, the first Ab is catumaxomab. In embodiments, the first Ab is efalizumab. In embodiments, the first Ab is toitumomab-1131. In embodiments, the first Ab is palivizumab. In embodiments, the first Ab is basilixumab. In embodiments, the first Ab is daclizumab. In embodiments, the first Ab is abciximab. In embodiments, the first Ab is murononomab. In embodiments, the first Ab is certolizumab. In embodiments, the first Ab is benralizumab. In embodiments, the first Ab is dupilumab. In embodiments, the first Ab is omalizumab. In embodiments, the first Ab is muromonab-CD3. In embodiments, the first Ab is AER002. In embodiments, the first Ab is durvalumab.

In embodiments, the first antibody is not tixagevimab. In embodiments, the first Ab is not cilgavimab. In embodiments, the first Ab is not pembrolizumab. In embodiments, the first Ab is not nivolumab. In embodiments, the first Ab is not bevacizumab. In embodiments, the first Ab is not ocrelizumab. In embodiments, the first Ab is not rituximab. In embodiments, the first Ab is not daratumumab. In embodiments, the first Ab is not pertuzumab. In embodiments, the first Ab is not trastuzumab. In embodiments, the first Ab is not infliximab. In embodiments, the first Ab is not tocilizumab. In embodiments, the first Ab is not atezolizumab. In embodiments, the first Ab is not tositumomab. In embodiments, the first Ab is not olaratumab. In embodiments, the first Ab is not rituximab. In embodiments, the first Ab is not basiliximab. In embodiments, the first Ab is not ibritumomab tiuxetan. In embodiments, the first Ab is not cetuximab. In embodiments, the first Ab is not natalizumab. In embodiments, the first Ab is not panitumumab. In embodiments, the first Ab is not ranibizumab. In embodiments, the first Ab is not eculizumab. In embodiments, the first Ab is not ofatumumab. In embodiments, the first Ab is not belimumab. In embodiments, the first Ab is not ipilimumab. In embodiments, the first Ab is not pertuzumab. In embodiments, the first Ab is not raxibacumab. In embodiments, the first Ab is not obinutuzumab. In embodiments, the first Ab is not siltuximab. In embodiments, the first Ab is not ramucirumab. In embodiments, the first Ab is not vedolizumab. In embodiments, the first Ab is not alemtuzumab. In embodiments, the first Ab is not necitumumab. In embodiments, the first Ab is not dinutuximab. In embodiments, the first Ab is not elotuzumab. In embodiments, the first Ab is not reslizumab. In embodiments, the first Ab is not bezlotoxumab. In embodiments, the first Ab is not obiltoxaximab. In embodiments, the first Ab is not avelumab. In embodiments, the first Ab is not sotrovimab. In embodiments, the first Ab is not bebtelovimab. In embodiments, the first Ab is not casirivirimab. In embodiments, the first Ab is not imdevimab. In embodiments, the first Ab is not etesevimab. In embodiments, the first Ab is not bamlanivimab. In embodiments, the first Ab is not amubarvimab. In embodiments, the first Ab is not regdanvimab. In embodiments, the first Ab is not romlusevimab. In embodiments, the first Ab is not adintrevimab. In embodiments, the first Ab is not 002-S21F2. In embodiments, the first Ab is not AZD3152. In embodiments, the first Ab is not lenzilumab. In embodiments, the first Ab is not ravulizumab. In embodiments, the first Ab is not canakinumab. In embodiments, the first Ab is not adalimumab. In embodiments, the first Ab is not sarilumab. In embodiments, the first Ab is not ronapreve. In embodiments, the first Ab is not vilobelimab. In embodiments, the first Ab is not casirivimab. In embodiments, the first Ab is not sarilumab. In embodiments, the first Ab is not leronlimab. In embodiments, the first Ab is not golimumab. In embodiments, the first Ab is not tabalumab. In embodiments, the first Ab is not veltuzumab. In embodiments, the first Ab is not mepolizumab. In embodiments, the first Ab is not secukinumab. In embodiments, the first Ab is not evolocumab. In embodiments, the first Ab is not blinatumomab. In embodiments, the first Ab is not adotrastuzumab. In embodiments, the first Ab is not brentuximab. In embodiments, the first Ab is not ipilumumab. In embodiments, the first Ab is not denosumab. In embodiments, the first Ab is not ustekinumab. In embodiments, the first Ab is not catumaxomab. In embodiments, the first Ab is not efalizumab. In embodiments, the first Ab is not toitumomab-1131. In embodiments, the first Ab is not palivizumab. In embodiments, the first Ab is not basilixumab. In embodiments, the first Ab is not daclizumab. In embodiments, the first Ab is not abciximab. In embodiments, the first Ab is not murononomab. In embodiments, the first Ab is not certolizumab. In embodiments, the first Ab is not benralizumab. In embodiments, the first Ab is not dupilumab. In embodiments, the first Ab is not omalizumab. In embodiments, the first Ab is not muromonab-CD3. In embodiments, the first Ab is not AER002. In embodiments, the first Ab is not durvalumab.

In embodiments, the first antibody is tixagevimab or cilgavimab. In some embodiments, the first antibody is tixagevimab. In other embodiments, the first antibody is cilgavimab.

In embodiments, the first antibody is a IgG1 monoclonal antibody. In embodiments, the first antibody is a fully human IgG1 monoclonal antibody. In embodiments, the first antibody is a IgG1 monoclonal antibody designed to target the spike protein of SARS-CoV-2. In embodiments, the first antibody is a fully human IgG1 monoclonal antibody designed to target the spike protein of SARS-CoV-2.

In embodiments, the first antibody is pemivibart (e.g., PEMGARDA®).

In embodiments, the first antibody is sipavibart (e.g., KAVIGALE®).

In embodiments, the first antiviral agent and the first antibody together provide synergistic effects, or are selected to provide synergistic effects.

b. First Antiviral, First Antibody, and Second Antiviral

In embodiments, a pharmaceutical combination comprises: i) a first antiviral agent, or a pharmaceutically acceptable salt thereof; ii) a first antibody; and iii) a second antiviral agent, or a pharmaceutically acceptable salt thereof. In such embodiments, a combination may comprise any of the combinations above, further comprising a second antiviral agent.

In embodiments, the second antiviral agent is a protease inhibitor. In embodiments, the second antiviral agent is an Mpro inhibitor. In embodiments, the second antiviral agent is an RdRp inhibitor. In embodiments, the second antiviral agent is an ACE2 decoy receptor drug.

In embodiments, the second antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir,

In embodiments, the second antiviral agent is selected from the group consisting of molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), and acyclovir.

In embodiments, the second antiviral agent is ritonavir. In embodiments, the second antiviral agent is nirmatrelvir. In embodiments, the second antiviral agent is remdesivir. In embodiments, the second antiviral agent is amprenavir. In embodiments, the second antiviral agent is atazanavir. In embodiments, the second antiviral agent is darunavir. In embodiments, the second antiviral agent is fosamprenavir. In embodiments, the second antiviral agent is indinavir. In embodiments, the second antiviral agent is lopinavir. In embodiments, the second antiviral agent is nelfinavir. In embodiments, the second antiviral agent is saquinavir. In embodiments, the second antiviral agent is tipranavir. In embodiments, the second antiviral agent is asunaprevir. In embodiments, the second antiviral agent is boceprevir. In embodiments, the second antiviral agent is grazoprevir. In embodiments, the second antiviral agent is glecaprevir. In embodiments, the second antiviral agent is paritaprevir. In embodiments, the second antiviral agent is simeprevir. In embodiments, the second antiviral agent is telaprevir. In embodiments, the second antiviral agent is molnupiravir. In embodiments, the second antiviral agent is GC376. In embodiments, the second antiviral agent is arbidol (umifenovir). In embodiments, the second antiviral agent is SSAA09E2. In embodiments, the second antiviral agent is ribavirin. In embodiments, the second antiviral agent is favilavir. In embodiments, the second antiviral agent is oseltamivir. In embodiments, the second antiviral agent is zanamivir. In embodiments, the second antiviral agent is abacavir. In embodiments, the second antiviral agent is stavudine. In embodiments, the second antiviral agent is valganciclovir. In embodiments, the second antiviral agent is ganciclovir. In embodiments, the second antiviral agent is cidofovir. In embodiments, the second antiviral agent is entecavir. In embodiments, the second antiviral agent is lamivudine. In embodiments, the second antiviral agent is maraviroc. In embodiments, the second antiviral agent is azidothymidine. In embodiments, the second antiviral agent is nelfinavir. In embodiments, the second antiviral agent is dolutegravir. In embodiments, the second antiviral agent is olsetamivir. In embodiments, the second antiviral agent is ensitrelvir (Xocova). In embodiments, the second antiviral agent is simnotrelvir. In embodiments, the second antiviral agent is bemnifosbuvir (AT-527). In embodiments, the second antiviral agent is deuremidevir (VV116). In embodiments, the second antiviral agent is azvudine. In embodiments, the second antiviral agent is sofosbuvir. In embodiments, the second antiviral agent is obeldesivir (GS-5245). In embodiments, the second antiviral agent is acyclovir. In embodiments, the second antiviral agent is obeldesivir.

In embodiments, the second antiviral agent is ensitrelvir.

In embodiments, the second antiviral agent is not a protease inhibitor. In embodiments, the second antiviral agent is not an Mpro inhibitor. In embodiments, the second antiviral agent is not an RdRp inhibitor. In embodiments, the second antiviral agent is not an ACE2 decoy receptor drug.

In embodiments, the second antiviral agent is not ritonavir. In embodiments, the second antiviral agent is not nirmatrelvir. In embodiments, the second antiviral agent is not remdesivir. In embodiments, the second antiviral agent is not amprenavir. In embodiments, the second antiviral agent is not atazanavir. In embodiments, the second antiviral agent is not darunavir. In embodiments, the second antiviral agent is not fosamprenavir. In embodiments, the second antiviral agent is not indinavir. In embodiments, the second antiviral agent is not lopinavir. In embodiments, the second antiviral agent is not nelfinavir. In embodiments, the second antiviral agent is not saquinavir. In embodiments, the second antiviral agent is not tipranavir. In embodiments, the second antiviral agent is not asunaprevir. In embodiments, the second antiviral agent is not boceprevir. In embodiments, the second antiviral agent is not grazoprevir. In embodiments, the second antiviral agent is not glecaprevir. In embodiments, the second antiviral agent is not paritaprevir. In embodiments, the second antiviral agent is not simeprevir. In embodiments, the second antiviral agent is not telaprevir. In embodiments, the second antiviral agent is not molnupiravir. In embodiments, the second antiviral agent is not GC376. In embodiments, the second antiviral agent is not arbidol (umifenovir). In embodiments, the second antiviral agent is not SSAA09E2. In embodiments, the second antiviral agent is not ribavirin. In embodiments, the second antiviral agent is not favilavir. In embodiments, the second antiviral agent is not oseltamivir. In embodiments, the second antiviral agent is not zanamivir. In embodiments, the second antiviral agent is not abacavir. In embodiments, the second antiviral agent is not stavudine. In embodiments, the second antiviral agent is not valganciclovir. In embodiments, the second antiviral agent is not ganciclovir. In embodiments, the second antiviral agent is not cidofovir. In embodiments, the second antiviral agent is not entecavir. In embodiments, the second antiviral agent is not lamivudine. In embodiments, the second antiviral agent is not maraviroc. In embodiments, the second antiviral agent is not azidothymidine. In embodiments, the second antiviral agent is not nelfinavir. In embodiments, the second antiviral agent is not dolutegravir. In embodiments, the second antiviral agent is not olsetamivir. In embodiments, the second antiviral agent is not ensitrelvir (Xocova). In embodiments, the second antiviral agent is not simnotrelvir. In embodiments, the second antiviral agent is not bemnifosbuvir (AT-527). In embodiments, the second antiviral agent is not deuremidevir (VV116). In embodiments, the second antiviral agent is not azvudine. In embodiments, the second antiviral agent is not sofosbuvir. In embodiments, the second antiviral agent is not obeldesivir (GS-5245). In embodiments, the second antiviral agent is not acyclovir.

In embodiments, the second antiviral agent is an antiviral agent from any of the lists of “additional agents having antiviral activity” in the section on additional active agents.

In embodiments, the second antiviral agent is ritonavir or nirmatrelvir. In some embodiments, the second antiviral agent is ritonavir. In other embodiments, the second antiviral agent is nirmatrelvir.

In embodiments, the first antiviral agent is nirmatrelvir, or a pharmaceutically acceptable salt thereof; and the second antiviral agent is ritonavir, or a pharmaceutically acceptable salt thereof.

In embodiments, the first antiviral agent, the first antibody, and the second antiviral agent together provide synergistic effects, or are selected to provide synergistic effects.

c. First Antiviral, First Antibody, Second Antiviral, and Second Antibody

In embodiments, the pharmaceutical combination comprises: i) a first antiviral agent, or a pharmaceutically acceptable salt thereof; ii) a first antibody; iii) a second antiviral agent, or a pharmaceutically acceptable salt thereof; and iv) a second antibody. In such embodiments, a combination may comprise any of the combinations above, further comprising a second antibody.

In embodiments, the second antibody (as shorthand, “second Ab”) is a monoclonal antibody. In embodiments, the second Ab is a polyclonal antibody. In embodiments, the second Ab is an IgA antibody. In embodiments, the second Ab is an IgD antibody. In embodiments, the second Ab is an IgE antibody. In embodiments, the second Ab is an IgG antibody. In embodiments, the second Ab is an IgM antibody. In embodiments, the second Ab is an anti-BlyS antibody. In some preferred embodiments, the first antibody is an IgG antibody. In embodiments, the second Ab is a broadly-neutralizing antibody. In embodiments, the second Ab is a broadly-neutralizing monoclonal antibody. In embodiments, the second Ab is a long-acting antibody. In embodiments, the second Ab is a long-acting monoclonal antibody. In embodiments, the second Ab is an anti-spike antibody. In embodiments, the second Ab is an IgG1 or IgG3 antibody. In embodiments, the second Ab is an IgG2 or IgG4 antibody.

In embodiments, the second antibody is not an IgA antibody. In embodiments, the second Ab is not an IgD antibody. In embodiments, the second Ab is not an IgE antibody. In embodiments, the second Ab is not an IgG antibody. In embodiments, the second Ab is not an IgM antibody. In embodiments, the second Ab is not an anti-BlyS antibody. In embodiments, the second Ab is not a broadly-neutralizing antibody. In embodiments, the second Ab is not a broadly-neutralizing monoclonal antibody. In embodiments, the second Ab is not a long-acting antibody. In embodiments, the second Ab is not a long-acting monoclonal antibody. In embodiments, the second Ab is not an anti-spike antibody. In embodiments, the second Ab is not an IgG1 or IgG3 antibody. In embodiments, the second Ab is not an IgG2 or IgG4 antibody.

In embodiments, the second antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casirivirimab, imdevimab, etesevimab, bamlanivimab, amubarvimab, regdanvimab, romlusevimab, adintrevimab, 002-S21F2, and durvalumab.

In embodiments, the second antibody is selected from the group consisting of AZD3152, Lenzilumab, Ravulizumab, Canakinumab, Adalimumab, Sarilumab, Ronapreve, Vilobelimab, Casirivimab, Sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, and AER002.

In embodiments, the second antibody is tixagevimab. In embodiments, the second Ab is cilgavimab. In embodiments, the second Ab is pembrolizumab. In embodiments, the second Ab is nivolumab. In embodiments, the second Ab is bevacizumab. In embodiments, the second Ab is ocrelizumab. In embodiments, the second Ab is rituximab. In embodiments, the second Ab is daratumumab. In embodiments, the second Ab is pertuzumab. In embodiments, the second Ab is trastuzumab. In embodiments, the second Ab is infliximab. In embodiments, the second Ab is tocilizumab. In embodiments, the second Ab is atezolizumab. In embodiments, the second Ab is tositumomab. In embodiments, the second Ab is olaratumab. In embodiments, the second Ab is rituximab. In embodiments, the second Ab is basiliximab. In embodiments, the second Ab is ibritumomab tiuxetan. In embodiments, the second Ab is cetuximab. In embodiments, the second Ab is natalizumab. In embodiments, the second Ab is panitumumab. In embodiments, the second Ab is ranibizumab. In embodiments, the second Ab is eculizumab. In embodiments, the second Ab is ofatumumab. In embodiments, the second Ab is belimumab. In embodiments, the second Ab is ipilimumab. In embodiments, the second Ab is pertuzumab. In embodiments, the second Ab is raxibacumab. In embodiments, the second Ab is obinutuzumab. In embodiments, the second Ab is siltuximab. In embodiments, the second Ab is ramucirumab. In embodiments, the second Ab is vedolizumab. In embodiments, the second Ab is alemtuzumab. In embodiments, the second Ab is necitumumab. In embodiments, the second Ab is dinutuximab. In embodiments, the second Ab is elotuzumab. In embodiments, the second Ab is reslizumab. In embodiments, the second Ab is bezlotoxumab. In embodiments, the second Ab is obiltoxaximab.

In embodiments, the second Ab is avelumab. In embodiments, the second Ab is sotrovimab. In embodiments, the second Ab is bebtelovimab. In embodiments, the second Ab is casivirimab. In embodiments, the second Ab is imdevimab. In embodiments, the second Ab is etesevimab. In embodiments, the second Ab is bamlanivimab. In embodiments, the second Ab is amubarvimab. In embodiments, the second Ab isregdanvimab. In embodiments, the second Ab is romlusevimab. In embodiments, the second Ab is adintrevimab. In embodiments, the second Ab is 002-S21F2. In embodiments, the second Ab is AZD3152. In embodiments, the second Ab is lenzilumab. In embodiments, the second Ab is ravulizumab. In embodiments, the second Ab is canakinumab. In embodiments, the second Ab is adalimumab. In embodiments, the second Ab is sarilumab. In embodiments, the second Ab is ronapreve. In embodiments, the second Ab is vilobelimab. In embodiments, the second Ab is casirivimab. In embodiments, the second Ab is sarilumab. In embodiments, the second Ab is leronlimab. In embodiments, the second Ab is golimumab. In embodiments, the second Ab is tabalumab. In embodiments, the second Ab is veltuzumab. In embodiments, the second Ab is mepolizumab. In embodiments, the second Ab is secukinumab. In embodiments, the second Ab is evolocumab. In embodiments, the second Ab is blinatumomab. In embodiments, the second Ab is adotrastuzumab. In embodiments, the second Ab is brentuximab. In embodiments, the second Ab is ipilumumab. In embodiments, the second Ab is denosumab. In embodiments, the second Ab is ustekinumab. In embodiments, the second Ab is catumaxomab. In embodiments, the second Ab is efalizumab. In embodiments, the second Ab is toitumomab-1131. In embodiments, the second Ab is palivizumab. In embodiments, the second Ab is basilixumab. In embodiments, the second Ab is daclizumab. In embodiments, the second Ab is abciximab. In embodiments, the second Ab is murononomab. In embodiments, the second Ab is certolizumab. In embodiments, the second Ab is benralizumab. In embodiments, the second Ab is dupilumab. In embodiments, the second Ab is omalizumab. In embodiments, the second Ab is muromonab-CD3. In embodiments, the second Ab is AER002. In embodiments, the second Ab is durvalumab.

In embodiments, the second antibody is not tixagevimab. In embodiments, the second Ab is not cilgavimab. In embodiments, the second Ab is not pembrolizumab. In embodiments, the second Ab is not nivolumab. In embodiments, the second Ab is not bevacizumab. In embodiments, the second Ab is not ocrelizumab. In embodiments, the second Ab is not rituximab. In embodiments, the second Ab is not daratumumab. In embodiments, the second Ab is not pertuzumab. In embodiments, the second Ab is not trastuzumab. In embodiments, the second Ab is not infliximab. In embodiments, the second Ab is not tocilizumab. In embodiments, the second Ab is not atezolizumab. In embodiments, the second Ab is not tositumomab. In embodiments, the second Ab is not olaratumab. In embodiments, the second Ab is not rituximab. In embodiments, the second Ab is not basiliximab. In embodiments, the second Ab is not ibritumomab tiuxetan. In embodiments, the second Ab is not cetuximab. In embodiments, the second Ab is not natalizumab. In embodiments, the second Ab is not panitumumab. In embodiments, the second Ab is not ranibizumab. In embodiments, the second Ab is not eculizumab. In embodiments, the second Ab is not ofatumumab. In embodiments, the second Ab is not belimumab. In embodiments, the second Ab is not ipilimumab. In embodiments, the second Ab is not pertuzumab. In embodiments, the second Ab is not raxibacumab. In embodiments, the second Ab is not obinutuzumab. In embodiments, the second Ab is not siltuximab. In embodiments, the second Ab is not ramucirumab. In embodiments, the second Ab is not vedolizumab. In embodiments, the second Ab is not alemtuzumab. In embodiments, the second Ab is not necitumumab. In embodiments, the second Ab is not dinutuximab. In embodiments, the second Ab is not elotuzumab. In embodiments, the second Ab is not reslizumab. In embodiments, the second Ab is not bezlotoxumab. In embodiments, the second Ab is not obiltoxaximab. In embodiments, the second Ab is not avelumab. In embodiments, the second Ab is not sotrovimab. In embodiments, the second Ab is not bebtelovimab. In embodiments, the second Ab is not casivirimab. In embodiments, the second Ab is not imdevimab. In embodiments, the second Ab is not etesevimab. In embodiments, the second Ab is not bamlanivimab. In embodiments, the second Ab is not amubarvimab. In embodiments, the second Ab is not regdanvimab. In embodiments, the second Ab is not romlusevimab. In embodiments, the second Ab is not adintrevimab. In embodiments, the second Ab is not 002-S21F2. In embodiments, the second Ab is not AZD3152. In embodiments, the second Ab is not lenzilumab. In embodiments, the second Ab is not ravulizumab. In embodiments, the second Ab is not canakinumab. In embodiments, the second Ab is not adalimumab. In embodiments, the second Ab is not sarilumab. In embodiments, the second Ab is not ronapreve. In embodiments, the second Ab is not vilobelimab. In embodiments, the second Ab is not casirivimab. In embodiments, the second Ab is not sarilumab. In embodiments, the second Ab is not leronlimab. In embodiments, the second Ab is not golimumab. In embodiments, the second Ab is not tabalumab. In embodiments, the second Ab is not veltuzumab. In embodiments, the second Ab is not mepolizumab. In embodiments, the second Ab is not secukinumab. In embodiments, the second Ab is not evolocumab. In embodiments, the second Ab is not blinatumomab. In embodiments, the second Ab is not adotrastuzumab. In embodiments, the second Ab is not brentuximab. In embodiments, the second Ab is not ipilumumab. In embodiments, the second Ab is not denosumab. In embodiments, the second Ab is not ustekinumab. In embodiments, the second Ab is not catumaxomab. In embodiments, the second Ab is not efalizumab. In embodiments, the second Ab is not toitumomab-1131. In embodiments, the second Ab is not palivizumab. In embodiments, the second Ab is not basilixumab. In embodiments, the second Ab is not daclizumab. In embodiments, the second Ab is not abciximab. In embodiments, the second Ab is not murononomab. In embodiments, the second Ab is not certolizumab. In embodiments, the second Ab is not benralizumab. In embodiments, the second Ab is not dupilumab. In embodiments, the second Ab is not omalizumab. In embodiments, the second Ab is not muromonab-CD3. In embodiments, the second Ab is not AER002. In embodiments, the second Ab is not durvalumab.

In embodiments, the second antibody is tixagevimab or cilgavimab. In some embodiments, the second antibody is tixagevimab. In other embodiments, the second antibody is cilgavimab.

In embodiments, the first antibody is tixagevimab or cilgavimab. In some embodiments, the first antibody is tixagevimab. In other embodiments, the first antibody is cilgavimab.

In embodiments, the second antibody is a IgG1 monoclonal antibody. In embodiments, the second antibody is a fully human IgG1 monoclonal antibody. In embodiments, the second antibody is a IgG1 monoclonal antibody designed to target the spike protein of SARS-CoV-2. In embodiments, the second antibody is a fully human IgG1 monoclonal antibody designed to target the spike protein of SARS-CoV-2.

In embodiments, the second antibody is pemivibart (e.g., PEMGARDA®).

In embodiments, the second antibody is sipavibart (e.g., KAVIGALE®).

In embodiments, the first antibody (“first Ab”) and the second antibody (“second Ab”) are both monoclonal antibodies. In embodiments, the first Ab is a monoclonal antibody and the second Ab is a polyclonal antibody. In other embodiments, the first Ab is a polyclonal antibody and the second Ab is a monoclonal antibody. In embodiments, the first Ab and the second Ab are both polyclonal antibodies. In embodiments, the first Ab and the second Ab are both broadly-neutralizing antibodies. In embodiments, the first Ab and the second Ab are both broadly-neutralizing monoclonal antibodies. In embodiments, the first Ab and the second Ab are both broadly-neutralizing polyclonal antibodies. In embodiments, the first Ab and the second Ab are both long-acting antibodies. In embodiments, the first Ab and the second Ab are both long-acting monoclonal antibodies. In embodiments, the first Ab and the second Ab are both long-acting polyclonal antibodies. In embodiments, the first Ab and the second Ab are not both monoclonal antibodies. In embodiments, the first Ab and the second Ab are not both polyclonal antibodies. In embodiments, the first Ab and the second Ab are not both broadly-neutralizing antibodies. In embodiments, the first Ab and the second Ab are not both broadly-neutralizing monoclonal antibodies. In embodiments, the first Ab and the second Ab are not both broadly-neutralizing polyclonal antibodies. In embodiments, the first Ab and the second Ab are not both long-acting antibodies. In embodiments, the first Ab and the second Ab are not both long-acting monoclonal antibodies. In embodiments, the first Ab and the second Ab are not both long-acting polyclonal antibodies.

In embodiments, the first antibody is tixagevimab and the second antibody is cilgavimab. In embodiments, the first antiviral agent is nirmatrelvir, or a pharmaceutically acceptable salt thereof; the second antiviral agent is ritonavir, or a pharmaceutically acceptable salt thereof; the first antibody is tixagevimab; and the second antibody is cilgavimab.

In embodiments, the first antiviral agent, or a pharmaceutically acceptable salt thereof, and the second antiviral agent, or a pharmaceutically acceptable salt thereof, are provided as separate pharmaceutical compositions. In embodiments, the first antiviral agent, or a pharmaceutically acceptable salt thereof, and the second antiviral agent, or a pharmaceutically acceptable salt thereof, are provided in the same pharmaceutical composition. In some preferred embodiments, wherein the first antiviral agent is nirmatrelvir, or a pharmaceutically acceptable salt thereof; and the second antiviral agent is ritonavir, or a pharmaceutically acceptable salt thereof; the nirmatrelvir and the ritonavir are provided together in the same antiviral composition. In some such embodiments, the antiviral composition is PAXLOVID™.

In embodiments, the first antibody and the second antibody are provided as separate pharmaceutical compositions. In embodiments, the first antibody and the second antibody are provided in the same pharmaceutical composition. In embodiments, wherein the first antibody is tixagevimab and the second antibody is cilgavimab, the tixagevimab and the cilgavimab are provided together in the same antibody composition. In some such embodiments, the antibody composition is EVUSHELD™.

In some embodiments, the pharmaceutical combination comprises an effective amount of PAXLOVID™ and an effective amount of EVUSHELD™.

In embodiments, the first antiviral agent, the first antibody, the second antiviral agent, and the second antibody together provide synergistic effects, or are selected to provide synergistic effects.

In some embodiments, wherein a pharmaceutical combination comprises two or more antibodies, the pharmaceutical combination provides broader neutralizing ability and reduces the possibility of viral variant antigenic escape in future strains, the measurement of which will be understood by those of skill.

d. Combinations Comprising an Additional Active Agent

In embodiments, a pharmaceutical combination will comprise an additional active agent. In embodiments, the pharmaceutical combination may comprise any of the combinations above, further comprising the additional active agent.

In embodiments, the pharmaceutical combination comprises: i) a first antiviral agent, or a pharmaceutically acceptable salt thereof; and ii) an additional active agent. In embodiments, the pharmaceutical combination comprises: i) a first antibody; and ii) an additional active agent. In embodiments, the pharmaceutical combination comprises: i) a first antiviral agent, or a pharmaceutically acceptable salt thereof; ii) a first antibody; and iii) an additional active agent. In embodiments, the pharmaceutical combination comprises: i) a first antiviral agent, or a pharmaceutically acceptable salt thereof; ii) a second antiviral agent, or a pharmaceutically acceptable salt thereof; and iii) an additional active agent. In embodiments, the pharmaceutical combination comprises: i) a first antibody; ii) a second antibody; and iii) an additional active agent. In embodiments, the pharmaceutical combination comprises: i) a first antiviral agent, or a pharmaceutically acceptable salt thereof; ii) a first antibody; iii) a second antiviral agent, or a pharmaceutically acceptable salt thereof; and iv) an additional active agent. In embodiments, the pharmaceutical combination comprises: i) a first antiviral agent, or a pharmaceutically acceptable salt thereof; ii) a first antibody; iii) a second antibody; and iv) an additional active agent. In embodiments, the pharmaceutical combination comprises: i) a first antiviral agent, or a pharmaceutically acceptable salt thereof; ii) a first antibody; iii) a second antiviral agent, or a pharmaceutically acceptable salt thereof; iv) a second antibody; and v) an additional active agent.

For example, in embodiments, the additional active agent is an interferon. Exemplary embodiments comprising an interferon as the additional active agent follow, and like embodiments will be readily appreciated for other additional active agents. In embodiments, the pharmaceutical combination comprises: i) a first antiviral agent, or a pharmaceutically acceptable salt thereof; and ii) an interferon. In embodiments, the pharmaceutical combination comprises: i) a first antibody; and ii) an interferon. In embodiments, the pharmaceutical combination comprises: i) a first antiviral agent, or a pharmaceutically acceptable salt thereof; ii) a first antibody; and iii) an interferon. In embodiments, the pharmaceutical combination comprises: i) a first antiviral agent, or a pharmaceutically acceptable salt thereof; ii) a second antiviral agent, or a pharmaceutically acceptable salt thereof; and iii) an interferon. In embodiments, the pharmaceutical combination comprises: i) a first antibody; ii) a second antibody; and iii) an interferon. In embodiments, the pharmaceutical combination comprises: i) a first antiviral agent, or a pharmaceutically acceptable salt thereof; ii) a first antibody; iii) a second antiviral agent, or a pharmaceutically acceptable salt thereof; and iv) an interferon. In embodiments, the pharmaceutical combination comprises: i) a first antiviral agent, or a pharmaceutically acceptable salt thereof; ii) a first antibody; iii) a second antibody; and iv) an interferon. In embodiments, the pharmaceutical combination comprises: i) a first antiviral agent, or a pharmaceutically acceptable salt thereof; ii) a first antibody; iii) a second antiviral agent, or a pharmaceutically acceptable salt thereof; iv) a second antibody; and v) an interferon.

In embodiments, the additional active agent, together with any one or more of the first antiviral agent, the first antibody, the second antiviral agent, or the second antibody, where each are present, provides synergistic effects, or is selected to provide synergistic effects. In embodiments, the additional active agent, and any one or more of the first antiviral agent, the first antibody, the second antiviral agent, or the second antibody, where each are present, together provide synergistic effects, or are selected to provide synergistic effects.

e. Additional Features of Certain Exemplary Embodiments

In embodiments, a pharmaceutical combination may be further described or known by the absence of one or more pharmaceutical agents, such as in certain embodiments above, and in other embodiments below or elsewhere herein. In embodiments, the pharmaceutical combination does not comprise remdesivir. In embodiments, the pharmaceutical combination does not comprise nirmatrelvir. In embodiments, the pharmaceutical combination does not comprise ritonavir. In embodiments, the pharmaceutical combination does not comprise molnupiravir. In embodiments, the pharmaceutical combination does not comprise remdesivir, nirmatrevir, ritonavir, or molnupiravir. In embodiments, the pharmaceutical combination does not comprise remdesivir, nirmatrelvir, or ritonavir. In embodiments, the pharmaceutical combination does not comprise remdesivir, ritonavir, or molnupiravir. In embodiments, the pharmaceutical combination does not comprise remdesivir, nirmatrelvir, or molnupiravir. In embodiments, the pharmaceutical combination does not comprise nirmatrelvir, ritonavir, or molnupiravir. In embodiments, the pharmaceutical combination does not comprise remdesivir or nirmatrelvir. In embodiments, the pharmaceutical combination does not comprise remdesivir or ritonavir. In embodiments, the pharmaceutical combination does not comprise remdesivir or molnupiravir. In embodiments, the pharmaceutical combination does not comprise nirmatrelvir or ritonavir. In embodiments, the pharmaceutical combination does not comprise nirmatrelvir or molnupiravir. In embodiments, the pharmaceutical combination does not comprise ritonavir or remdesivir. In embodiments, the pharmaceutical combination does not comprise ritonavir or molnupiravir.

### D. PHARMACEUTICAL COMPOSITIONS

In embodiments, any one of the first antiviral agent, or a pharmaceutically acceptable salt thereof, the second antiviral agent, or a pharmaceutically acceptable salt thereof, the first antibody, the second antibody, or any combination thereof, may be administered to a subject as a pharmaceutical composition or multiple pharmaceutical compositions. In embodiments, the first antiviral agent, or a pharmaceutically acceptable salt thereof, and the second antiviral agent, or a pharmaceutically acceptable salt thereof, are comprised in the same pharmaceutical composition. In embodiments, the first antibody and the second antibody are comprised in the same pharmaceutical composition.

For example, in embodiments, a disclosed method comprises administering to the patient a first pharmaceutical composition comprising the first antiviral agent, or a pharmaceutically acceptable salt thereof, and the second antiviral agent, or a pharmaceutically acceptable salt thereof; and a second pharmaceutical composition comprising a first antibody and optionally, a second antibody. As another example, a disclosed method comprises administering to the patient a first pharmaceutical composition comprising a first antiviral agent, or a pharmaceutically acceptable salt thereof; a second pharmaceutical composition comprising a second antiviral agent, or a pharmaceutically acceptable salt thereof; and a third pharmaceutical composition comprising a first antibody and a second antibody.

Such pharmaceutical compositions can be formulated as or into any suitable dosage form, such as aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilised formulations, tablets, capsules, pills, powders, pulsatile release formulations, multi-particulate formulations, immediate release, controlled release, sustained release, extended release, and modified release formulations, and mixed immediate release and controlled release formulations.

In embodiments, a pharmaceutical composition can be incorporated into a variety of formulations, e.g., pharmaceutically acceptable vehicles, for therapeutic administration. For example, in embodiments, the antiviral agents or monoclonal antibodies of the present disclosure can be formulated into pharmaceutical compositions by formulation with one or more pharmaceutically acceptable carriers, diluents, and/or excipients, and may be formulated into preparations in solid, semi-solid, liquid, or gaseous forms, such as tablets, capsules, powders, granules, ointments (e.g., skin creams), solutions, suppositories, injections, inhalants, and aerosols. As such, administration of the antiviral agents or monoclonal antibodies can be achieved in various ways, for example, oral, buccal, rectal, parenteral, intraperitoneal, intradermal, transdermal, intracheal, intravenous, or intramuscular, administration.

For oral preparations, any one of the first antiviral agent, or a pharmaceutically acceptable salt thereof, the second antiviral agent, or a pharmaceutically acceptable salt thereof, the first antibody, the second antibody, or any combination thereof, can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

For intravenous or intramuscular administration, any one of the first antiviral agent, or a pharmaceutically acceptable salt thereof, the second antiviral agent, or a pharmaceutically acceptable salt thereof, the first antibody, the second antibody, or any combination thereof, can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some preferred embodiments, an antibody is formulated for delivery by intramuscular injection. In embodiments, an antibody is administered by intramuscular injection. In embodiments, an antibody is not formulated for delivery by IV infusion. In embodiments, an antibody is not administered by IV infusion.

Unit dosage forms for oral administration such as syrups, elixirs, and suspensions may be provided wherein each dosage unit, for example, teaspoonful, tablespoonful, tablet or suppository, contains a predetermined amount of any one of the first antiviral agent, or a pharmaceutically acceptable salt thereof, the second antiviral agent, or a pharmaceutically acceptable salt thereof, the first antibody, the second antibody, or any combination thereof. Similarly, unit dosage forms for injection or intravenous administration may comprise any one of the first antiviral agent, or a pharmaceutically acceptable salt thereof, the second antiviral agent, or a pharmaceutically acceptable salt thereof, the first antibody, the second antibody, or any combination thereof, in a composition as a solution in sterile water, normal saline or another pharmaceutically acceptable carrier.

As used herein, the phrase “unit dosage form” refers to a physically discrete unit suited as unitary dosages for the patient to be treated, each unit containing a predetermined quantity of active agent calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilised state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a patient for an extended or brief period of time.

In embodiments, any one of the first antiviral agent, or a pharmaceutically acceptable salt thereof, the second antiviral agent, or a pharmaceutically acceptable salt thereof, the first antibody, the second antibody, or any combination thereof, is formulated in a pharmaceutically acceptable oral dosage form, including oral solid dosage forms and oral liquid dosage forms. In embodiments, the compositions are formulated as a pharmaceutically acceptable oral solid dosage form, including lozenges, troches, tablets, capsules, caplets, powders, pellets, multiparticulates, beads, spheres, capsules, pills, and/or any combinations thereof. Oral solid dosage forms may be formulated as immediate release, controlled release, sustained release, extended release, or modified release formulations. In embodiments, a disclosed antibody is formulated for oral administration by means of gastric auto-injector, as known for example by reference to Abramson A, et al. Nat Biotechnol. 2022 January; 40(1):103-109, incorporated by reference herein.

In embodiments, solid dosage forms may comprise pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. In embodiments, solid dosage forms may comprise pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s), including preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the pharmaceutical formulation, and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid (ALA).

As many formulations and dosage forms will be known to those of skill for various of the disclosed pharmaceutical agents, one of skill will appreciate how to select such formulations and dosage forms for use in a disclosed pharmaceutical combination or method, as well as how to prepare additional formulations or dosage forms of an agent, based on the general knowledge in the art and the information in this disclosure.

In embodiments, the first antiviral agent, or a pharmaceutically acceptable salt thereof, or the second antiviral agent, or a pharmaceutically acceptable salt thereof, the first antibody, the second antibody, or any combination thereof, may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, at least 3.0 mg/kg, at least 3.1 mg/kg, at least 3.2 mg/kg, at least 3.3 mg/kg, at least 3.4 mg/kg, at least 3.5 mg/kg, at least 3.6 mg/kg, at least 3.7 mg/kg, at least 3.8 mg/kg, at least 3.9 mg/kg, or at least 4.0 mg/kg, or at least 5.0 mg/kg, as well as amounts within these ranges.

In embodiments, the first antiviral agent, or a pharmaceutically acceptable salt thereof, or the second antiviral agent, or a pharmaceutically acceptable salt thereof, the first antibody, the second antibody, or any combination thereof, may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, at least 250 mg, at least 275 mg, at least 300 mg, at least 325 mg, at least 350 mg, at least 400 mg, or at least 500 mg, as well as amounts within these ranges.

In embodiments, wherein a disclosed method or combination comprises an additional active compound, or pharmaceutically acceptable salt thereof, the additional active compound, or pharmaceutically acceptable salt thereof, may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, at least 3.1 mg/kg, at least 3.2 mg/kg, at least 3.3 mg/kg, at least 3.4 mg/kg, at least 3.5 mg/kg, at least 3.6 mg/kg, at least 3.7 mg/kg, at least 3.8 mg/kg, at least 3.9 mg/kg, at least 4.0 mg/kg, or at least 5.0 mg/kg, as well as amounts within these ranges.

In embodiments, wherein a disclosed method or combination comprises an additional active compound, or pharmaceutically acceptable salt thereof, the additional active compound, or pharmaceutically acceptable salt thereof, may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, at least 275 mg, at least 300 mg, at least 325 mg, or at least 350 mg, at least 400 mg, or at least 500 mg, as well as amounts within these ranges.

As many dosage forms and dose amounts will be known to those of skill for various of the disclosed pharmaceutical agents, one of skill will appreciate how to prepare or select other dosage forms or dose amounts for use in a disclosed pharmaceutical combination or method, as well as how to prepare or select additional dosage forms or dose amounts of an agent, based on knowledge in the art and this disclosure.

As an example, in embodiments wherein the first antiviral agent is nirmatrelvir, or a pharmaceutically acceptable salt thereof, and wherein the second antiviral agent is ritonavir, or a pharmaceutically acceptable salt thereof, the dose amount for a subject may be 300 mg of nirmatrelvir and 100 mg of ritonavir. In embodiments, one dose may be administered to a subject at the same time each morning, and one dose may be administered at the same time each evening. In embodiments, one dose comprises two tablets of nirmatrelvir and one tablet of ritonavir. In other embodiments, the dose amount for a subject may be 150 mg of nirmatrelvir and 100 mg of ritonavir. In embodiments, one dose may be administered to a subject at the same time each morning, and one dose may be administered at the same time each evening. In embodiments, one dose comprises one tablet of nirmatrelvir and one tablet of ritonavir. Other such available dosage forms and standard dose amounts will be known to those of skill, for example by reference to a package insert, storage, dosing, and administration guide, or similar informational material, such as from a manufacturer, and such available dosage forms and standard dose amounts may be used in some embodiments where no other dosage form or standard dose amount is known by reference to the disclosure herein. As another example, in embodiments wherein the first antibody is tixagevimab, and the second antibody is cilgavimab, the dose amount for a subject may be 300 mg of cilgavimab and 300 mg of tixagevimab, administered via intramuscular injection. In other embodiments, the dose amount for a subject may be 150 mg of cilgavimab and 150 mg of tixagevimab, administered via intramuscular injection.

### E. PHARMACEUTICAL KITS

In some aspects are disclosed pharmaceutical kits comprising the disclosed pharmaceutical combinations, for example prepared as one or more pharmaceutical compositions, and which may be used in the disclosed methods.

In embodiments, a pharmaceutical kit comprises one or more antiviral agents or monoclonal antibodies described herein, or a composition comprising one or more antiviral agents or monoclonal antibodies described herein. In embodiments, the kit comprises an antiviral agent described herein, or a pharmaceutically acceptable salt thereof. In one embodiment, the kit comprises the first antiviral agent described herein, or a pharmaceutically acceptable salt thereof. In one embodiment, the kit comprises the second antiviral agent described herein, or a pharmaceutically acceptable salt thereof. In one embodiment, the kit comprises both the first antiviral agent described herein, or a pharmaceutically acceptable salt thereof, and the second antiviral agent described herein, or a pharmaceutically acceptable salt thereof. In embodiments, the kit comprises a monoclonal antibody described herein. In one embodiment, the kit comprises the first antibody described herein. In one embodiment, the kit comprises the second antibody described herein. In one embodiment, the kit comprises both the first antibody described herein and the second antibody described herein. In embodiments, the kit comprises both the first antiviral agent described herein, or a pharmaceutically acceptable salt thereof, and the second antiviral agent described herein, or a pharmaceutically acceptable salt thereof; and both the first antibody and the second antibody.

Kits generally comprise suitable packaging. The kits may comprise one or more containers comprising any agent or composition described herein. Each component (if there is more than one component) can be packaged in separate containers or some components can be combined in one container where cross-reactivity and shelf life permit. Kits may comprise components in unit dosage forms, in bulk packages (e.g., multi-dose packages), or in sub-unit doses. For example, kits may be provided that contain sufficient dosages of a antiviral agent or monoclonal antibody as disclosed herein and/or an additional pharmaceutically active compound to provide effective treatment of a subject for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the antiviral agents and monoclonal antibodies and instructions for use and be packaged in quantities sufficient for storage and use in pharmacies (e.g., hospital pharmacies and compounding pharmacies).

Kits may optionally include a set of instructions, which may be printed instructions, packaged with the kit, or a reference to electronic instructions, such as on a website or in a mobile or smartphone app, provide for example with a website link, QR code, or application name. The instructions included with a kit generally will include information as to the components thereof and their administration to a patient. Kits may include, for example, a package insert, a storage, dosing, and administration guide, or the like.

As pharmaceutical kits will be known to those of skill for various of the disclosed pharmaceutical agents, and which may include one, two, or more pharmaceutical agents co-packaged together, one of skill will appreciate how to prepare other such pharmaceutical kits, comprising the disclosed pharmaceutical combinations, and for use in a disclosed method, based on the general knowledge in the art and the information in this disclosure.

For example, in embodiments wherein the first antibody is tixagevimab and the second antibody is cilgavimab, the first and second antibodies may be co-packaged as a pharmaceutical kit, wherein the kit comprises a carton comprising two vials, one of each antibody: a vial comprising tixagevimab solution for injection, and a vial comprising cilgavimab solution for injection, both to be administered by intramuscular injection, and both at a concentration of 150 mg of antibody per 1.5 mL, which a subject is to be administered as separate, consecutive intramuscular injections, which may be at different injection sites, 150 mg of tixagevimab and 150 mg of cilgavimab. The vials are identified by white versus dark gray vial cap.

For example, in embodiments wherein the first antiviral agent is nirmatrelvir, or a pharmaceutically acceptable salt thereof, and wherein the second antiviral agent is ritonavir, or a pharmaceutically acceptable salt thereof, the first and second antiviral agents may be formulated in solid dosage forms. In such embodiments, a unit dosage form may comprise a standard dose pack or a reduced dose pack. In such embodiments, the standard dose pack comprises three tablets. In such embodiments, the reduced dose pack comprises two tablets. In such embodiments, a unit dosage form contains the first and second antiviral agents in solid dosage form, such as in the dosage form of a tablet. In such embodiments, multiple unit dosage forms may be packaged together. For example, in such embodiments, each pharmaceutical kit comprises five blister cards with the morning and evening doses of the first and second antiviral agents.

### F. METHODS OF TREATMENT

In some aspects are disclosed methods of treating a subject having PASC or one or more symptoms of PASC, using the disclosed pharmaceutical compositions and disclosed pharmaceutical kits.

In embodiments, the method comprises administering to a subject an effective amount of a combination of: i) a first antiviral agent, or a pharmaceutically acceptable salt thereof; and ii) a first antibody.

In embodiments, the method comprises administering to a subject an effective amount of a combination of: i) a first antiviral agent, or a pharmaceutically acceptable salt thereof; ii) a first antibody; and iii) a second antiviral agent, or a pharmaceutically acceptable salt thereof.

In embodiments, the method comprises administering to a subject an effective amount of a combination of: i) a first antiviral agent, or a pharmaceutically acceptable salt thereof; ii) a first antibody; iii) a second antiviral agent, or a pharmaceutically acceptable salt thereof; and iv) a second antibody.

In some preferred embodiments, the method comprises administering to a subject an effective amount of a combination of: nirmatrelvir, or a pharmaceutically acceptable salt thereof; ritonavir, or a pharmaceutically acceptable salt thereof; tixagevimab; and cilgavimab.

In some preferred embodiments, the method comprises administering to a subject an effective amount of PAXLOVID™ and an effective amount of EVUSHELD™.

In embodiments, a disclosed method further comprises administering to a subject an additional active agent, such as an effective amount of an additional active agent. In embodiments, the method comprises administering to a subject an effective amount of a combination of: i) a first antiviral agent, or a pharmaceutically acceptable salt thereof; and ii) an additional active agent. In embodiments, the method comprises administering to a subject an effective amount of a combination of: i) a first antibody; and ii) an additional active agent. In embodiments, the method comprises administering to a subject an effective amount of a combination of: i) a first antiviral agent, or a pharmaceutically acceptable salt thereof; ii) a first antibody; and iii) an additional active agent. In embodiments, the method comprises administering to a subject an effective amount of a combination of: i) a first antiviral agent, or a pharmaceutically acceptable salt thereof; ii) a second antiviral agent, or a pharmaceutically acceptable salt thereof; and iii) an additional active agent. In embodiments, the method comprises administering to a subject an effective amount of a combination of: i) a first antibody; ii) a second antibody; and iii) an additional active agent. In embodiments, the method comprises administering to a subject an effective amount of a combination of: i) a first antiviral agent, or a pharmaceutically acceptable salt thereof; ii) a first antibody; iii) a second antiviral agent, or a pharmaceutically acceptable salt thereof; and iv) an additional active agent. In embodiments, the method comprises administering to a subject an effective amount of a combination of: i) a first antiviral agent, or a pharmaceutically acceptable salt thereof; ii) a first antibody; iii) a second antibody; and iv) an additional active agent. In embodiments, the method comprises administering to a subject an effective amount of a combination of: i) a first antiviral agent, or a pharmaceutically acceptable salt thereof; ii) a first antibody; iii) a second antiviral agent, or a pharmaceutically acceptable salt thereof; iv) a second antibody; and v) an additional active agent.

In embodiments, the additional active agent is an interferon. Exemplary embodiments comprising an interferon as the additional active agent follow, and like embodiments will be readily appreciated for other additional active agents. In embodiments, the method comprises administering to a subject an effective amount of a combination of: i) a first antiviral agent, or a pharmaceutically acceptable salt thereof; and ii) an interferon. In embodiments, the method comprises administering to a subject an effective amount of a combination of: i) a first antibody; and ii) an interferon. In embodiments, the method comprises administering to a subject an effective amount of a combination of: i) a first antiviral agent, or a pharmaceutically acceptable salt thereof; ii) a first antibody; and iii) an interferon. In embodiments, the method comprises administering to a subject an effective amount of a combination of: i) a first antiviral agent, or a pharmaceutically acceptable salt thereof; ii) a second antiviral agent, or a pharmaceutically acceptable salt thereof; and iii) an interferon. In embodiments, the method comprises administering to a subject an effective amount of a combination of: i) a first antibody; ii) a second antibody; and iii) an interferon. In embodiments, the method comprises administering to a subject an effective amount of a combination of: i) a first antiviral agent, or a pharmaceutically acceptable salt thereof; ii) a first antibody; iii) a second antiviral agent, or a pharmaceutically acceptable salt thereof; and iv) an interferon. In embodiments, the method comprises administering to a subject an effective amount of a combination of: i) a first antiviral agent, or a pharmaceutically acceptable salt thereof; ii) a first antibody; iii) a second antibody; and iv) an interferon. In embodiments, the method comprises administering to a subject an effective amount of a combination of: i) a first antiviral agent, or a pharmaceutically acceptable salt thereof; ii) a first antibody; iii) a second antiviral agent, or a pharmaceutically acceptable salt thereof; iv) a second antibody; and v) an interferon.

a. Manner of Administration

In embodiments, wherein a method of treating a subject having PASC comprises administering to a subject an effective amount of a first antiviral agent, or a pharmaceutically acceptable salt thereof; optionally, a second antiviral agent, or a pharmaceutically acceptable salt thereof; a first antibody; and optionally, a second antibody; as well as further comprising, optionally, an additional active agent; each such antiviral agent, antibody, and additional active agent, where present, or the composition comprising it, may be administered to the subject separately, sequentially, or simultaneously. Herein, as shorthand, any antiviral agent, antibody, or additional active agent, where present, in a combination, may be referred to as a “pharmaceutical agent.” Where one or more pharmaceutical agents are formulated together, such formulation herein as shorthand may be referred to as a “pharmaceutical composition.”

In embodiments, “sequential” administration refers to administration of a pharmaceutical agent or pharmaceutical composition immediately following administration of another pharmaceutical agent or pharmaceutical composition (e.g., within about 5 minutes of the administration of the first pharmaceutical agent or pharmaceutical composition). In embodiments, “simultaneous” administration refers to administration of a pharmaceutical agent or pharmaceutical composition at substantially the same time as another pharmaceutical agent or pharmaceutical composition. In embodiments, “separate” administration refers to administration of a pharmaceutical agent or pharmaceutical composition and at least one other pharmaceutical agent or pharmaceutical composition, with time elapsing between the administrations.

In embodiments wherein two or more pharmaceutical agents or pharmaceutical compositions are administered separately, such administration may include elapsed time between each administration of between about 5 minutes to about 30 minutes, about 10 minutes to about 60 minutes, about 30 minutes to about 180 minutes, about 180 minutes to about 360 minutes, or more than 360 minutes, such as 8 hours, 12 hours, 16 hours, 20 hours, 24 hours, 36 hours, 48 hours, 72 hours, 5 days, 7 days, 10 days, 14 days, 21 days, 30 days, and any such durations in between.

In embodiments, administration may comprise separate, sequential, or simultaneous oral administrations, sublingual administrations, buccal administrations, intravenous injections, intra-arterial injections, intraperitoneal injections, intraosseous injections, intramuscular injections, intrathecal injections, intracerebroventricular injections, rectal administrations, vaginal administrations, ocular administrations, nasal administrations, cutaneous administrations, topical administrations, otic administrations, transdermal administrations, or a combination thereof, as would be apparent to one of skill depending on the desired therapeutic effect. Further, as mentioned, in embodiments, a patient is administered the pharmaceutical agents and/or pharmaceutical compositions of a disclosed combination through more than one administration, wherein each administration is not the same means of administration.

In embodiments, a pharmaceutical agent or pharmaceutical composition is administered separately from at least one other pharmaceutical agent or pharmaceutical composition. In embodiments, two or more pharmaceutical agents or pharmaceutical compositions are administered sequentially. In embodiments, two or more pharmaceutical agents or pharmaceutical compositions are administered simultaneously.

In embodiments, wherein a method of treating a subject having PASC comprises administering to the subject an effective amount of a first antiviral agent, or a pharmaceutically acceptable salt thereof, and an effective amount of a first antibody, the first antiviral agent, or a pharmaceutically acceptable salt thereof, may be administered to the subject prior to treatment with the first antibody, simultaneously with treatment with the first antibody, or after treatment with the first antibody.

In embodiments, administration to a subject of an effective amount of a first antiviral agent, or a pharmaceutically acceptable salt thereof, “prior to” treatment with a first antibody refers to administration to the subject of an effective amount of a first antiviral agent, or a pharmaceutically acceptable salt thereof, occurring before the administration to the subject of treatment with an effective amount of a first antibody. In embodiments, administration to a subject of an effective amount of a first antiviral agent, or a pharmaceutically acceptable salt thereof, may occur immediately prior to treatment with an effective amount of a first antibody. For example, in embodiments, administration to a subject of an effective amount of a first antiviral agent, or a pharmaceutically acceptable salt thereof, immediately prior to treatment, occurs within about 5 minutes of treatment with an effective amount of a first antibody. In other embodiments, “prior to” treatment refers to separate administration where the elapsed time between each administration is greater than about 5 minutes, for example between about 5 minutes and about one hour, between about one hour and one day, between about one day and one week, or between about one week and one month.

In embodiments, administration to a subject of an effective amount of a first antiviral agent, or a pharmaceutically acceptable salt thereof, “simultaneously” with treatment with a first antibody refers to administration to a subject of an effective amount of a first antiviral agent, or a pharmaceutically acceptable salt thereof, at substantially the same time as administration to the subject of treatment with a first antibody.

In embodiments, administration to a subject of an effective amount of a first antiviral agent, or a pharmaceutically acceptable salt thereof, “after” treatment with a first antibody refers to administration to the subject of an effective amount of a first antiviral agent, or a pharmaceutically acceptable salt thereof, occurring following the administration to the subject of treatment with an effective amount of a first antibody. In embodiments, administration to a subject of an effective amount of a first antiviral agent, or a pharmaceutically acceptable salt thereof, may occur immediately after treatment with an effective amount of a first antibody. In embodiments, administration to a subject of an effective amount of a first antiviral agent, or a pharmaceutically acceptable salt thereof, immediately after treatment, occurs within about 5 minutes of treatment with an effective amount of a first antibody. In other embodiments, “after” treatment refers to separate administration where the elapsed time between each administration is greater than about 5 minutes, for example between about 5 minutes and about one hour, between about one hour and one day, between about one day and one week, or between about one week and one month.

In embodiments, the first antiviral agent, or a pharmaceutically acceptable salt thereof, is administered prior to treatment with the first antibody; or the first antiviral agent, or a pharmaceutically acceptable salt thereof, is administered after treatment with the first antibody; or the first antiviral agent, or a pharmaceutically acceptable salt thereof, or is administered simultaneously with treatment with the first antibody. In embodiments, the first antiviral agent, or a pharmaceutically acceptable salt thereof, is administered prior to treatment with the first antibody. In other embodiments, the first antiviral agent, or a pharmaceutically acceptable salt thereof, is administered after treatment with the first antibody. In yet other embodiments, the first antiviral agent, or a pharmaceutically acceptable salt thereof, is administered simultaneously with treatment with the first antibody.

In embodiments, the second antiviral agent, or a pharmaceutically acceptable salt thereof, is administered prior to treatment with the first antibody; or the second antiviral agent, or a pharmaceutically acceptable salt thereof, is administered after treatment with the first antibody; or the second antiviral agent, or a pharmaceutically acceptable salt thereof, is administered simultaneously with treatment with the first antibody. In embodiments, the second antiviral agent, or a pharmaceutically acceptable salt thereof, is administered prior to treatment with the first antibody. In other embodiments, the second antiviral agent, or a pharmaceutically acceptable salt thereof, is administered after treatment with the first antibody. In yet other embodiments, the second antiviral agent, or a pharmaceutically acceptable salt thereof, is administered simultaneously with treatment with the first antibody.

In embodiments, the first antiviral agent, or a pharmaceutically acceptable salt thereof, is administered prior to treatment with the second antibody; or the first antiviral agent, or a pharmaceutically acceptable salt thereof, is administered after treatment with the second antibody; or the first antiviral agent, or a pharmaceutically acceptable salt thereof, is administered simultaneously with treatment with the second antibody. In embodiments, the first antiviral agent, or a pharmaceutically acceptable salt thereof, is administered prior to treatment with the second antibody. In other embodiments, the first antiviral agent, or a pharmaceutically acceptable salt thereof, is administered after treatment with the second antibody. In yet other embodiments, the first antiviral agent, or a pharmaceutically acceptable salt thereof, is administered simultaneously with treatment with the second antibody.

In embodiments, the second antiviral agent, or a pharmaceutically acceptable salt thereof, is administered prior to treatment with the second antibody; or the second antiviral agent, or a pharmaceutically acceptable salt thereof, is administered after treatment with the second antibody; or the second antiviral agent, or a pharmaceutically acceptable salt thereof, is administered simultaneously with treatment with the second antibody. In embodiments, the second antiviral agent, or a pharmaceutically acceptable salt thereof, is administered prior to treatment with the second antibody. In other embodiments, the second antiviral agent, or a pharmaceutically acceptable salt thereof, is administered after treatment with the second antibody. In yet other embodiments, the second antiviral agent, or a pharmaceutically acceptable salt thereof, is administered simultaneously with treatment with the second antibody.

In embodiments, the first antiviral agent, or a pharmaceutically acceptable salt thereof, and the second antiviral agent, or a pharmaceutically acceptable salt thereof, are both administered prior to treatment with the first and/or second antibody; or the first antiviral agent, or a pharmaceutically acceptable salt thereof, and the second antiviral agent, or a pharmaceutically acceptable salt thereof, are both administered after treatment with the first and/or second antibody; or the first antiviral agent, or a pharmaceutically acceptable salt thereof, and the second antiviral agent, or a pharmaceutically acceptable salt thereof, are both administered simultaneously with treatment with the first and/or second antibody.

In embodiments, the first antiviral agent, or a pharmaceutically acceptable salt thereof, and the second antiviral agent, or a pharmaceutically acceptable salt thereof, are both administered prior to treatment with the first and/or second antibody. In embodiments, the first antiviral agent, or a pharmaceutically acceptable salt thereof, and the second antiviral agent, or a pharmaceutically acceptable salt thereof, are both administered after treatment with the first and/or second antibody. In embodiments, the first antiviral agent, or a pharmaceutically acceptable salt thereof, and the second antiviral agent, or a pharmaceutically acceptable salt thereof, are both administered simultaneously with treatment with the first and/or second antibody.

In embodiments, the disclosed pharmaceutical combinations and compositions can be administered by a variety of routes including oral, mucosal (e.g., buccal, sublingual), rectal, transdermal, subcutaneous, intravenous, intramuscular, inhaled, and intranasal. Such compositions can be prepared in a manner known in the pharmaceutical art (see, e.g., Adejare A, ed. Remington. 2020), and in general will be known by reference to the active agent(s) present in a combination or composition, and the desired delivery thereof.

For example, in embodiments wherein the first antiviral agent is nirmatrelvir, or a pharmaceutically acceptable salt thereof, and wherein the second antiviral agent is ritonavir, or a pharmaceutically acceptable salt thereof, a solid dosage form comprising nirmatrelvir, such as a tablet, and a separate solid dosage form comprising ritonavir, such as a tablet, may be administered to a subject orally. In embodiments, the first antiviral agent and the second antiviral agent are co-formulated as a single dosage form, such as a unit dosage form, such as a fixed-dose combination (FDC). In one such example embodiment, nirmatrelvir, or a pharmaceutically acceptable salt thereof, and ritonavir, or a pharmaceutically acceptable salt thereof, are co-formulated together into a single solid dosage form, such as a tablet, comprising both antiviral agents.

In an example, in embodiments wherein the first antibody is tixagevimab and the second antibody is cilgavimab, an injection of tixagevimab and an injection of cilgavimab may be administered consecutively, in any order, via IM injection, at two separate injection sites (e.g., into the gluteal or buttocks muscles). In embodiments, the first antibody and the second antibody are co-formulated as a single dosage form. In one such example embodiment, tixagevimab and cilgavimab are co-formulated together into a single injectable dosage form, such as a formulation for intramuscular administration, comprising both antibodies.

b. Dosage

In embodiments, the disclosed pharmaceutical combinations and compositions are administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Dosage levels thus may differ from patient to patient, for individual patients across time, and for different pharmaceutical compositions and formulations, but shall be able to be determined with ordinary skill.

Determination of appropriate dosing shall include not only the determination of single dosage amounts, but also the determination of the number and timing of doses, and the time(s) of day or time(s) preferable for administration. Dose and dosage may vary depending upon the onset, progression, severity, frequency, duration, probability of, or susceptibility of the symptom to which treatment is directed, clinical endpoint desired, previous, simultaneous or subsequent treatments, general health, age, gender, and race of the patient, bioavailability, potential adverse systemic, regional, or local side effects, the presence of other disorders or diseases in the patient, and other factors appreciated by those in the art (e.g., medical or familial history). Dose amount, frequency, or duration may be increased or reduced, as indicated by the clinical outcome desired, status of the disorder or symptoms, any adverse side effects of the treatment or therapy, or concomitant medications. Those in the art will appreciate the factors that may influence the dosage, frequency, and timing required to provide an amount sufficient or effective for providing a therapeutic effect or benefit, and to do so depending on the type of therapeutic effect desired and to avoid or minimize adverse effects. In embodiments, the dose actually administered will be determined by a physician, in light of the relevant circumstances, including the disorder to be treated, the chosen route of administration, the actual composition administered, the age, weight, and response of the individual patient, and the severity of the patient's symptoms, and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In general, the disclosed methods will be appreciated to work for any subject, although individual variation is to be expected, and will be appreciated in view of the disclosure and ordinary skill. Where there is variation between individuals, modification to the disclosed combinations, compositions, and methods will be understood based on the teachings herein in combination with general knowledge in the art. In some instances, certain personalized or precision medicine approaches may be utilized, based on individual characteristics, including variations in drug metabolism (e.g., related to the metabolism of any one or more agents in a combination), genetics, or immune system (e.g., differences for immunocompromised subjects).

In embodiments, the genetic variation is a phenotype which can be associated with susceptibility to PASC or a symptom of PASC. In embodiments, a genetic variation is a variation associated with lung-related diseases, autoimmune diseases, or inflammatory diseases. In embodiments, a genetic variation can be attributed to a specific chromosomal locus. In embodiments, the locus is 3p21.31, ABO, PPP1R15A, SLC6A20, DPP9, FOXP4, TYK2, CXCR6, LZTFL1, IFNAR2, OAS1, OAS2, OAS3, or any combinations thereof. The term “genetic variation” refers herein to a change in a gene sequence relative to a reference sequence (e.g., a commonly-found and/or wild-type sequence). Genetic variation may be recombination events or mutations such as substitution/deletion/insertion events like point and splice site mutations.

In embodiments, a disclosed method reduces the severity of PASC or one or more symptoms thereof in a subject. In embodiments, the severity of PASC or a symptom thereof in a subject is quantified by one or more PASC biomarkers. PASC biomarkers may be detected from biological specimens, for example, a subject's blood, such as plasma or serum, or saliva. In embodiments, a PASC biomarker is assessed by measuring its serum concentration. In embodiments, a subject with PASC expresses a different biomarker profile than that of a healthy individual, than that of an individual recovered from PASC, and/or than that of an individual with acute COVID-19 infection. In embodiments, the level of a PASC biomarker in a subject suffering from PASC is higher than that of a healthy individual, that of an individual recovered from PASC, and/or that of an individual with acute COVID-19 infection. In embodiments, the level of a PASC biomarker in a subject suffering from PASC is lower than that of a healthy individual, that of an individual recovered from PASC, and/or that of an individual with acute COVID-19 infection.

In embodiments, a disclosed method reduces the concentration of a PASC biomarker by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. In embodiments, a disclosed method increases the concentration of a PASC biomarker by at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. In embodiments, a disclosed method causes a change in the concentration of a PASC biomarker to become closer to a baseline level. “Baseline level” refers to the level of a biomarker observed in healthy populations not experiencing PASC. Baseline levels differ among biomarkers and will be known to those of skill, or can be measured by standard techniques (Lai Y J, et al. Front Med (Lausanne). 2023; 10:1085988; Yong S J, et al. Rev Med Virol. 2023; 33(2): e2424).

In embodiments, a PASC biomarker is an acute phase protein. Without being bound by theory, acute phase proteins are a class of proteins that undergo changes in concentration in blood plasma in response to inflammation or tissue damage. In embodiments, the acute phase protein PASC biomarker is albumin, Complement 5b-9 (C5b-9), C-reactive protein (CRP), ferritin, or fibrinogen.

In embodiments, a PASC biomarker is a biochemical marker. In embodiments, the biochemical marker is a ratio between two molecules. In embodiments, the biochemical PASC biomarker is 1-Methylnicotinamide, 2-Phenylphenol, 3,5-Dihydroxybenzoic acid, ADA (adenosine deaminase), alanine aminotransferase (ALT), aspartate aminotransferase (AST), β-glucan, CPA3, glutamine/glutamate ratio, indole-3-lactic acid, L-cysteine, lactate dehydrogenase (LDH), L-glutamine, L-methionine, ornithine, pipecolic acid, quinolinic acid, quinolinic acid/tryptophan, sarcosine, S-sulfocysteine, sulfotransferase 1A1 (ST1A1), taurine, tryptase, urokinase type plasminogen activator (uPA).

In embodiments, a PASC biomarker is a cytokine. Cytokines are small signaling proteins that coordinate the interactions of different cell types involved in the amplification and regulation of the inflammatory response. In embodiments, a cytokine is a chemokine. Chemokines are a class of cytokine that induce the movement of other cell types, such as toward a tissue injury site. In embodiments, the cytokine PASC biomarker is CCL2, CCL3, CCL4, CCL5, CCL7, CCL19, CCL20, CCL23, CXCL1, CXCL9, CXCL10, CXCL11, FIt3L, G-CSF, GM-CSF, IFN-α, IFN-β, IFN-γ, IL-1a, IL-1B, IL-2, IL-4, IL-6, IL-7, IL-10, IL-10RB, IL-12B, IL-13, IL-17, IL-18, IL-33, IP-10, M-CSF, SCF, TGF-α, TGF-β, TNF-α, TNF-β.

In embodiments, a PASC biomarker is a neurological biomarker. Without being bound by theory, neurological biomarkers are proteins that provide information about the function or condition of the nervous system. In embodiments, the neurological PASC biomarker is granulocyte colony stimulating factor (GDNF), glial cell derived neurotrophic factor (GFAP), nerve growth factor beta (NGF-β), neurofilament light chain (NFL), neurotrophin 3 (NT-3), plasma neurofilament light chain/plasma glial fibrillary acidic protein (pGFAP/pNFL).

In embodiments, a PASC biomarker is a cardiovascular biomarker. Without being bound by theory, cardiovascular biomarkers are proteins that provide information about the function or condition of the cardiovascular system. In embodiments, the cardiovascular protein PASC biomarker is Angiopoietin 2 (Ang-2), Collagen type I alpha 2 (Col1A2), Collagen type III alpha 1 (Col3A1), D-dimer, erythrocyte sedimentation rate (ESR), endothelin 1 (ET-1), antihemophilic factor (Factor VIII), Hemoglobin, Matrix metalloproteinase 1 (MMP-1), Matrix metalloproteinase 9 (MMP-9), Myeloperoxidase (MPO), nitric oxide (NO), platelet derived growth factor BB (PDGF-BB), soluble intercellular adhesion molecule 1 (SICAM-1), soluble thrombomodulin (sTM), soluble vascular endothelial growth factor receptor (sVEGFR), Soluble vascular cell adhesion molecule 1 (sVCAM-1), vascular endothelial growth factor (VEGF), von Willebrand factor antigen (VWF:Ag), von Willebrand factor propeptide (VWF:pp).

In embodiments, a PASC biomarker is any protein which is increased or decreased in concentration in sample taken from a subject relative to a baseline. In embodiments, the protein PASC biomarker is Anti-severe acute respiratory syndrome coronavirus 2 antibody (Ab), artemin (ARTN), alpha smooth muscle actin (α-SMA), AXIN1, caspase-8 (CASP-8), cystatin D (CST-5), cystatin C, high sensitivity troponin T (Hs TnT), insulin like growth factor binding protein 4 (IGFBP-4), lipopolysaccharide binding protein (LBP), miRNA21, myeloid related protein 8/14 (MRP8/14), neutrophil gelatinase associated lipocalin (NGAL), N terminal pro B type natriuretic peptide/N terminal B type natriuretic peptide (NT-proBNP/NT-BNP), osteoprotegerin (OPG), oncostatin M (OSM), sirtuin 2 (SIRT2), STAM binding protein (STAMBP), Tumor necrosis factor receptor superfamily member 9 (TNFRSF9), Zonulin.

In embodiments, the PASC biomarker is albumin. In embodiments, the PASC biomarker is CRP. In embodiments, the PASC biomarker is ferritin. In embodiments, the PASC biomarker is granulocyte colony-stimulating factor (G-CSF). In embodiments, the PASC biomarker is interferon alpha (IFN-α). In embodiments, the PASC biomarker is interleukin-1B (IL-1B). In embodiments, the PASC biomarker is interleukin-6 (IL-6). In embodiments, the PASC biomarker is interleukin-13 (IL-13). In embodiments, the PASC biomarker is interleukin-17 (IL-17). In embodiments, the PASC biomarker is tumor necrosis factor alpha (TNF-α). In embodiments, the PASC biomarker is D-dimer. In embodiments, the PASC biomarker is erythrocyte sedimentation rate (ESR). In embodiments, the PASC biomarker is hemoglobin.

c. Treatment of Symptoms of PASC

In some aspects are disclosed methods of treating a subject having post-acute sequelae of COVID-19 (PASC), or one or more symptoms thereof (“symptoms of PASC” or “PASC-associated symptoms”). As used herein, the term “treating” therefore may refer to reducing or alleviating one or more symptoms of PASC in a subject. Where “reducing” a symptom includes by an amount up to and including 100%, reducing also includes “eliminating” the symptom.

In embodiments, a disclosed method or combination reduces or alleviates a symptom of PASC by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. Those of skill in the art will know the appropriate symptoms and corresponding clinically measurable indicators associated with symptoms of PASC. The reduction or alleviation of a symptom includes reducing the incidence or severity of that particular symptom. PASC symptoms generally include neurological, cardiac, and/or pulmonary symptoms (Lai Y J, et al. Front Med (Lausanne). 2023; 10:1085988; Proal A D, VanElzakker M B. Front Microbiol. 2021; 12:698169).

Without being bound by theory, the symptoms of PASC are caused by several biological mechanisms. Without being bound by theory, one underlying cause may be the persistence of SARS-CoV-2 virus or viral RNA in tissue as viral reservoirs. Without being bound by theory, the persistence of replicating virus or viral RNA may continue to activate the host immune system, thereby causing the persistence of certain immune responses such as inflammation. Without being bound by theory, the immune response to low but consistent immune activation underlying PASC is different from the immune response to an acute COVID-19 infection. Without being bound by theory, the difference in immune responses results in different pathology, symptoms, and biomarker profiles between the two indications (Proal A D, et al. Nat Immunol. 2023; 24(10):1616-1627). In embodiments, the disclosed method or combination supports the immune response associated with PASC to treat a subject having PASC or one or more symptoms thereof.

### Neurological Symptoms

In embodiments, the symptom of PASC is a neurological symptom. In embodiments, a disclosed method is useful for treating a neurological symptom of PASC. Neurological symptoms of PASC include sleep disorders, chronic headaches, olfactory disorders, taste disorders, brain fog, memory loss, decreased concentration, depression, anxiety, post-traumatic stress disorder (PTSD), dizziness, vertigo, tinnitus, hearing loss, instability, delirium, hallucinations, small fiber neuropathy, postural tremor, chronic pain, neurodegeneration, and myalgia (Takao M, Ohira M. Psychiatry Clin Neurosci. 2023; 77(2):72-83).

In embodiments, the neurological symptom is sleep disorders. In embodiments, the neurological symptom is chronic headaches. In embodiments, the neurological symptom is olfactory disorders. In embodiments, the neurological symptom is taste disorders. In embodiments, the neurological symptom is brain fog. In embodiments, the neurological symptom is memory loss. In embodiments, the neurological symptom is decreased concentration. In embodiments, the neurological symptom is depression. In embodiments, the neurological symptom is anxiety. In embodiments, the neurological symptom is post-traumatic stress disorder (PTSD). In embodiments, the neurological symptom is dizziness. In embodiments, the neurological symptom is vertigo. In embodiments, the neurological symptom is tinnitus. In embodiments, the neurological symptom is hearing loss. In embodiments, the neurological symptom is instability. In embodiments, the neurological symptom is delirium. In embodiments, the neurological symptom is hallucinations. In embodiments, the neurological symptom is small fiber neuropathy. In embodiments, the neurological symptom is postural tremor. In embodiments, the neurological symptom is chronic pain. In embodiments, the neurological symptom is neurodegeneration. In embodiments, the neurological symptom is myalgia.

In embodiments, treating the neurological symptom returns the level of a biomarker to within about 50%, 40%, 30%, 20%, 10%, 5%, or 1% of a baseline level. Neurological biomarkers include but are not limited to GDNF, GFAP, NGF-β, NFL, NT-3, and/or pGFAP/pNFL. In embodiments, treating the neurological symptom results in an increased serum GDNF concentration. In embodiments, treating the neurological symptom results in a decreased serum GFAP concentration. In embodiments, treating the neurological symptom results in an increased serum NGF-β concentration. In embodiments, treating the neurological symptom results in a decreased serum NT-3 concentration. In embodiments, treating the neurological symptom results in a decreased serum pGFAP/pNFL concentration (Lai Y J, et al. Front Med (Lausanne). 2023; 10:1085988).

### Cardiovascular Symptoms

In embodiments, the symptom of PASC is a cardiovascular symptom. In embodiments, a disclosed method is useful for treating a cardiovascular symptom of PASC. Cardiovascular symptoms of PASC include nonspecific chest pain, tightness in the chest, palpitations, tachycardia, conduction disturbances, rhythm disorders, orthostatic hypotension, vasovagal syncope, postural orthostatic tachycardia syndrome (POTS), phlebitis, and thrombophlebitis (Takao M, Ohira M. Psychiatry Clin Neurosci. 2023; 77(2):72-83).

In embodiments, the cardiovascular symptom (herein as shorthand, “CV symptom”) is nonspecific chest pain. In embodiments, the CV symptom is tightness in the chest. In embodiments, the CV symptom is palpitations. In embodiments, the CV symptom is tachycardia. In embodiments, the CV symptom is conduction disturbances. In embodiments, the CV symptom is rhythm disorders. In embodiments, the CV symptom is orthostatic hypotension. In embodiments, the CV symptom is vasovagal syncope. In embodiments, the CV symptom is postural orthostatic tachycardia syndrome. In embodiments, the CV symptom is phlebitis. In embodiments, the CV symptom is thrombophlebitis.

In embodiments, treating the cardiovascular symptom returns the level of a biomarker to within about 50%, 40%, 30%, 20%, 10%, 5%, or 1% of a baseline level. Cardiovascular biomarkers include Ang-2, Col1A2, Col3A1, D-dimer, ESR, ET-1, Factor VIII, Hemoglobin, MMP-1, MMP-9, MPO, NO, PDGF-BB, SICAM-1, STM, SVEGFR, SVCAM-1, VEGF, VWF:Ag, and/or VWF:pp. In embodiments, treating the cardiovascular (CV) symptom results in an increased serum Ang-2 concentration. In embodiments, treating the CV symptom results in a decreased serum Col1A2 concentration. In embodiments, treating the CV symptom results in a decreased serum Col3A1 concentration. In embodiments, treating the CV symptom results in a decreased serum D-dimer concentration. In embodiments, treating the CV symptom results in a decreased ESR. In embodiments, treating the CV symptom results in a decreased serum ET-1 concentration. In embodiments, treating the CV symptom results in a decreased serum Factor VIII concentration. In embodiments, treating the CV symptom results in an increased serum hemoglobin concentration. In embodiments, treating the CV symptom results in an increased serum MMP-1 concentration. In embodiments, treating the CV symptom results in a decreased serum MMP-9 concentration. In embodiments, treating the CV symptom results in a decreased serum MPO concentration. In embodiments, treating the CV symptom results in an increased serum NO concentration. In embodiments, treating the CV symptom results in a decreased serum PDGF-BB concentration. In embodiments, treating the CV symptom results in a decreased serum sICAM-1 concentration. In embodiments, treating the CV symptom results in a decreased serum sTM concentration. In embodiments, treating the CV symptom results in an increased serum sVEGFR concentration. In embodiments, treating the CV symptom results in a decreased serum sVCAM-1 concentration. In embodiments, treating the CV symptom results in a decreased serum VEGF concentration. In embodiments, treating the CV symptom results in a decreased serum VWF:Ag concentration. In embodiments, treating the CV symptom results in a decreased serum VWF:pp concentration (Lai Y J, et al. Front Med (Lausanne). 2023; 10:1085988).

### Pulmonary Symptoms

In embodiments, the symptom of PASC is a pulmonary symptom. In embodiments, a disclosed method is useful for treating a pulmonary symptom of PASC. Pulmonary symptoms of PASC include dyspnea, persistent cough, wheezing, worsening of asthma, decreased pulmonary diffusion capacity, persistent abnormal imaging findings, pleuritis, cough, sleep apnea, and sore throat (Takao M, Ohira M. Psychiatry Clin Neurosci. 2023; 77(2):72-83; Wang L, et al. J Biomed Inform. 2022; 125:103951).

In embodiments, the pulmonary symptom is dyspnea. In embodiments, the pulmonary symptom is a cough, including, in some such embodiments, a persistent cough. In embodiments, the pulmonary symptom is wheezing. In embodiments, the pulmonary symptom is worsening of asthma. In embodiments, the pulmonary symptom is decreased pulmonary diffusion capacity. In embodiments, the pulmonary symptom is persistent abnormal imaging findings. In embodiments, the pulmonary symptom is pleuritis. In embodiments, the pulmonary symptom is sleep apnea. In embodiments, the pulmonary symptom is a sore throat.

### Musculoskeletal Symptoms

In embodiments, the symptom of PASC is a musculoskeletal symptom. In embodiments, a disclosed method is useful for treating a musculoskeletal symptom of PASC. Musculoskeletal symptoms of PASC include arthritis, back pain, abnormal gait, and joint pain (Takao M, Ohira M. Psychiatry Clin Neurosci. 2023; 77(2):72-83; Wang L, et al. J Biomed Inform. 2022; 125:103951).

In embodiments, the musculoskeletal symptom is arthritis. In embodiments, the musculoskeletal symptom is back pain. In embodiments, the musculoskeletal symptom is abnormal gait. In embodiments, the musculoskeletal symptom is joint pain.

### Endocrinological Symptoms

In embodiments, the symptom of PASC is an endocrinological symptom. In embodiments, a disclosed method is useful for treating an endocrinological symptom of PASC. Endocrinological symptoms of PASC include impaired glucose metabolism, subacute thyrotoxicosis, Hashimoto's disease, Graves' disease, and dyslipidemia (Takao M, Ohira M. Psychiatry Clin Neurosci. 2023; 77(2):72-83).

In embodiments, the endocrinological symptom is impaired glucose metabolism. In embodiments, the endocrinological symptom is subacute thyrotoxicosis. In embodiments, the endocrinological symptom is Hashimoto's disease. In embodiments, the endocrinological symptom is Graves' disease. In embodiments, the endocrinological symptom is dyslipidemia.

### Dermal Symptoms

In embodiments, the symptom of PASC is a dermal symptom. In embodiments, a disclosed method is useful for treating a dermal symptom of PASC. Dermal symptoms of PAC include eruption, urticaria, skin lesion, telogen effluvium, nail changes, erythema, and trichodynia (Takao M, Ohira M. Psychiatry Clin Neurosci. 2023; 77(2):72-83; Wang L, et al. J Biomed Inform. 2022; 125:103951).

In embodiments, the dermal symptom is eruption. In embodiments, the dermal symptom is urticaria. In embodiments, the dermal symptom is skin lesion. In embodiments, the dermal symptom is telogen effluvium. In embodiments, the dermal symptom is nail changes. In embodiments, the dermal symptom is erythema. In embodiments, the dermal symptom is trichodynia.

### Kidney Symptoms

In embodiments, the symptom of PASC is a kidney symptom. In embodiments, a disclosed method is useful for treating a kidney symptom of PASC. Kidney symptoms of PASC include decreased globural filtration rate, urinary incontinence, and microscopic hematuria (Takao M, Ohira M. Psychiatry Clin Neurosci. 2023; 77(2):72-83; Wang L, et al. J Biomed Inform. 2022; 125:103951).

In embodiments, the kidney symptom is decreased glomerular filtration rate (GFR). In embodiments, the kidney symptom is urinary incontinence. In embodiments, the kidney symptom is decreased microscopic hematuria.

### Other Symptoms

In embodiments, symptoms of PASC include fever, chills, muscle ache, body ache, chest pain, stomach pain, nausea, vomiting, diarrhea. In embodiments, the symptom of PASC is gastroesophageal reflux, stress, swelling, bleeding, weight loss, weight gain, abdominal pain, constipation, pain in extremities, paresthesia, peripheral edema, itchiness, lymphadenopathy, and edema (Wang L, et al. J Biomed Inform. 2022; 125:103951).

In embodiments, symptoms of PASC include fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, and stomach pain. PASC or “long COVID” symptoms commonly include fatigue, headache, sleep problems (e.g., insomnia), dizziness, difficulty thinking or concentration (i.e., brain fog), change or loss in smell or taste, depression, and anxiety.

In embodiments, a disclosed method or combination reduces the incidence or severity of post-acute sequelae of COVID-19 (PASC)-associated fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, or stomach pain by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, or stomach pain.

In embodiments, a disclosed method or combination reduces the incidence or severity of PASC-associated fatigue, headache, sleep problems (e.g., insomnia), dizziness, difficulty thinking or concentration (i.e., brain fog), change in smell, change in taste, loss of smell, loss of taste, depression, or anxiety by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated fatigue, headache, sleep problems (e.g., insomnia), dizziness, difficulty thinking or concentration (i.e., brain fog), change in or loss of smell, change in or loss of taste, depression, or anxiety.

In embodiments, a disclosed method or combination reduces the incidence or severity of a symptom of PASC, such as any PASC symptom or “sequelae” herein, by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%, up to and including by 100%. In embodiments, a disclosed method or combination eliminates the symptom of PASC.

In embodiments, a disclosed method or combination reduces the incidence or severity of PASC-associated fatigue by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated fatigue.

In embodiments, a disclosed method or combination reduces the incidence or severity of PASC-associated headache by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated headache.

In embodiments, a disclosed method or combination reduces the incidence or severity of PASC-associated sleep problems by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated sleep problems.

In embodiments, a disclosed method or combination reduces the incidence or severity of PASC-associated dizziness by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated dizziness.

In embodiments, a disclosed method or combination reduces the incidence or severity of PASC-associated change or loss of smell by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated change or loss of smell.

In embodiments, a disclosed method or combination reduces the incidence or severity of PASC-associated change or loss of taste by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated change or loss of taste.

In embodiments, a disclosed method or combination reduces the incidence or severity of PASC-associated depression by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated depression.

In embodiments, a disclosed method or combination reduces the incidence or severity of PASC-associated anxiety by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated anxiety.

In embodiments, a disclosed method or combination reduces the incidence or severity of PASC-associated difficulty thinking or concentration (i.e., brain fog) by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates PASC-associated difficulty thinking or concentration (i.e., brain fog).

In embodiments, a symptom of PASC is a symptom associated with functional status or quality of life, a respiratory condition, a neurologic condition, a psychiatric condition, a symptom that affects exercise capacity, or a symptom that affects balance and fall risk. In embodiments, a disclosed method or combination reduces the incidence or severity of a PASC-associated symptom associated with functional status or quality of life, respiratory condition, neurologic condition, psychiatric condition, or symptom that affects balance and fall risk, by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a symptom of PASC associated with functional status or quality of life, respiratory condition, neurologic condition, psychiatric condition, or symptom that affects balance and fall risk.

In embodiments, a disclosed method or combination reduces the incidence or severity of a symptom of PASC associated with functional status or quality of life by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a symptom of PASC associated with functional status or quality of life.

In embodiments, a disclosed method or combination reduces the incidence or severity of a PASC-associated respiratory condition by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a PASC-associated respiratory condition.

In embodiments, a disclosed method or combination reduces the incidence or severity of a PASC-associated neurologic condition by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a PASC-associated neurologic condition.

In embodiments, a disclosed method or combination reduces the incidence or severity of a PASC-associated psychiatric condition by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a PASC-associated psychiatric condition.

In embodiments, a disclosed method or combination reduces the incidence or severity of a symptom of PASC that affects exercise capacity by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a symptom of PASC that affects exercise capacity.

In embodiments, a disclosed method or combination reduces the incidence or severity of a symptom of PASC that affects balance and fall risk by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a symptom of PASC that affects balance and fall risk.

PASC has been associated with numerous cytopathic effects, such as mitochondrial damage or dysfunction, oxidative stress, immune dysregulation, tissue damage, and cell death (Gonzalez-Garcia et al., Int. J. Mol. Sci. 2023, 24(9), 8290). In embodiments, a disclosed method or combination reduces the incidence or severity of a PASC-associated cytopathic effect by e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%. In embodiments, a disclosed method or combination eliminates a PASC-associated cytopathic effect.

In embodiments where a disclosed method or combination is used to treat PASC or a symptom of PASC, the disclosed method or combination will treat one or more “sequelae” such as defined above.

In embodiments, where a disclosed method or combination is used to treat PASC or a symptom of PASC, the disclosed method or combination is capable of one or more of: a) decreasing or preventing myalgia; b) decreasing or preventing fibromyalgia; c) decreasing or preventing idiopathic pulmonary fibrosis; d) decreasing or preventing fatigue; e) decreasing or preventing muscle fatigue; f) decreasing or preventing muscle dysfunction; g) decreasing or mitochondrial dysfunction; h) decreasing or preventing dyspnea after exertion; i) improving exercise tolerance or the ability to conduct activities of daily living; j) decreasing or preventing postural orthostatic tachycardia syndrome; or k) decreasing or preventing tachycardia; I) improving mitochondrial function; m) improving mitochondrial capacity or energetics (e.g., improvement in oxidative ATP synthesis following exercise or exertion); n) reducing oxidative stress and/or reducing reactive oxygen species (ROS); o) improving mood disorders and/or depression; p) improving cognitive function; q) increasing skeletal muscle or other organ vascular perfusion; r) improving endothelial function; s) decreasing or preventing diabetes (e.g., new onset diabetes); or t) improving inflammation.

While in embodiments, the disclosed methods and combinations are used to treat PASC, or at least one symptom of PASC, in other embodiments the methods and combinations are used to treat conditions(s), disease(s), syndrome(s), symptom(s), or disability(ies) that become manifest in a patient following a SARS-CoV-2 infection, past the standard incubation period, transmission period, or symptomatic period of a COVID infection, and which may be grouped together using different conceptual categories and/or terminology, some of which may be distinguishable from PASC or from the symptoms of PASC, or understood to be different from PASC or from the symptoms of PASC by those of skill. For example, in embodiments, the disclosed methods and combinations are used to treat post-COVID-19 conditions (PCC). In embodiments, the disclosed methods and combinations are used to treat long-haul COVID. In embodiments, the disclosed methods and combinations are used to treat prolonged COVID. In embodiments, the disclosed methods and combinations are used to treat chronic COVID. In embodiments, the disclosed methods and combinations are used to treat relapsing COVID. In embodiments, the disclosed methods and combinations are used to treat protracted COVID. In embodiments, the disclosed methods and combinations are used to treat post-COVID-19 syndrome. In yet other embodiments, the disclosed methods and combinations are used to treat PASC, or at least one symptom of PASC, but are not used to treat one or more of such other conditions(s), disease(s), syndrome(s), symptom(s), or disability(ies) above. For example, in embodiments, the disclosed methods and combinations are not used to treat post-COVID-19 conditions (PCC). In embodiments, the disclosed methods and combinations are not used to treat long-haul COVID. In embodiments, the disclosed methods and combinations are not used to treat prolonged COVID. In embodiments, the disclosed methods and combinations are not used to treat chronic COVID. In embodiments, the disclosed methods and combinations are not used to treat relapsing COVID. In embodiments, the disclosed methods and combinations are not used to treat protracted COVID. In embodiments, the disclosed methods and combinations are not used to treat post-COVID-19 syndrome.

Those of skill in the art will know the appropriate symptoms and corresponding clinically measurable indicators used to diagnose and/or assess symptoms of PASC. Herein, “assess” or “assessment” refer to a means or method used with a patient to measure, estimate, or evaluate a disorder, symptom, or characteristic of the patient, whether qualitatively or quantitatively, and whether performed by the therapist or other clinician (e.g., an interview), by the patient his or herself (e.g., a self-reported questionnaire), by a third-party or by a computer, including a medical device (e.g., as defined by FDA or another regulatory body) or other device (e.g., a medical sensor or biosensor, a watch or fitness tracker), and whether graded by a human decision-maker or an artificial intelligence, machine learning, or computer algorithm. Assessments may include self-report measures, patient-reported surveys, questionnaires, interviews, and the like.

Exemplary assessment tools for evaluating symptoms of PASC are shown below in Tables 1 and 2. These and other measures can also be found in the Centers for Disease Control and Prevention's health measures toolbox (see, e.g., Post-COVID Conditions: Information for Healthcare Providers, available at https://www.cdc.gov/coronavirus/2019-ncov/hcp/clinical-care/post-covid-conditions.html, Updated Sep. 22, 2022, and last accessed as of the filing date hereof), the American Academy of Physical Medicine & Rehabilitation's functional assessments, and assessment tools for other rehabilitation needs (e.g., bowel and bladder function, pain, activities of daily living, cognition, mobility, sleep). Assessment should be tailored to the subject's symptoms and presentation.

TABLE 1
Exemplary assessment tools for
evaluating subjects with PASC symptoms

| Category | Tools |
|---|---|
| Functional | Patient-Reported Outcomes Measurement Information |
| status or | System (PROMIS) (e.g., Cognitive Function 4a) |
| quality of life | Post-Covid-19 Functional Status Scale (PCFS) |
| | EuroQol-5D (EQ-5D) |
| Respiratory | Modified Medical Research Council (mMRC) |
| conditions | Dyspnea Scale |
| Neurologic | Montreal Cognitive Assessment (MoCA) |
| conditions | Mini Mental Status Examination (MMSE) |
| | Compass 31 (for dysautonomia) |
| | Neurobehavioral Symptom Inventory |
| Psychiatric | Generalized Anxiety Disorder-7 (GAD-7) |
| conditions | Patient Health Questionnaire-9 (PHQ-9) |
| | PTSD Symptom Scale (PSS) |
| | Screen for Posttraumatic Stress Symptoms (SPTSS) |
| | PTSD Checklist for DSM-5 (PCL-5) |
| | Impact of Event Scale-Revised (IESR) |
| | Hospital Anxiety and Depression Scale (HADS) |
| Other | Wood Mental Fatigue Inventory (WMFI) |
| conditions | Fatigue Severity Scale |
| | Insomnia Severity Index (ISI) |
| | Connective Tissue Disease Screening Questionnaire |

TABLE 2
Exemplary functional and other testing tools
for evaluating subjects with PASC symptoms

| Category | Tools |
|---|---|
| Exercise capacity | 1-minute sit-to-stand test |
| | 2-minute step test |
| | 10 Meter Walk Test (10MWT) |
| | 6-minute walk test |
| Balance and fall risk | BERG Balance Scale |
| | Tinetti Gait and Balance Assessment Tool |
| Other | Tilt-table testing (e.g., for POTS) |
| | Orthostatic HR assessment |

An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like). One or more other aspects of a psychosocial, behavioral, or drug-assisted therapy also may be “computer-assisted,” wherein one or more steps of such therapy involve the use of a computer in addition to or as a replacement for some work which would otherwise be performed by a therapist.

### G. EXAMPLES

The following examples are included for illustrative purposes only and are not intended to limit the scope of the invention, which shall be known by reference to the complete disclosure and appended claims.

### Example 1: Treating Patients Having PASC

PASC is diagnosed in patients according to diagnostic techniques known to those of skill, for example, by detection of SARS-CoV-2 virus (e.g., by real-time reverse transcriptase-polymerase chain reaction (RT-PCR)) during a time period of at least 28 days after the first positive PCR test, or otherwise from the date selected as the date of infection; or is diagnosed from the patients' medical histories, for example, because of the persistence or appearance of conditions(s), disease(s), syndrome(s), symptom(s), or disability(ies) following a SARS-CoV-2 infection that are most probably causally related to the SARS-CoV-2 infection.

Following PASC diagnosis, patients are treated according to a disclosed method. Specifically, the patient is administered an effective amount of a first antiviral agent, or a pharmaceutically acceptable salt thereof; optionally, a second antiviral agent, or a pharmaceutically acceptable salt thereof; a first antibody; and optionally, a second antibody. Exemplary treatments are as follows:

Patient 1: Patient 1 is administered a single dose of sotrovimab (500 mg) as a single intravenous dose. On the same day, Patient 1 begins taking a daily dose of 100 mg of remdesivir (by oral administration), which is continued for a total of five consecutive days. Patient 1 shows an improvement in at least one symptom of PASC or at least one biomarker of PASC.

Patient 2: Patient 2 is administered a single dose of tixagevimab and cilgavimab as a single 300 mg intramuscular dose (150 mg tixagevimab and 150 mg cilgavimab as a single injectable formulation). On the same day Patient 2 begins taking two daily doses of both nirmatrelvir and ritonavir tablets, co-packaged as Paxlovid™, in accordance with the package instructions. Specifically, Patient 2 takes a morning dose consisting of one 150 mg nirmatrelvir tablet and one 150 mg ritonavir tablet (both taken orally); and an evening dose consisting of one 150 mg nirmatrelvir tablet and one 150 mg ritonavir tablet (both taken orally). Patient 2 continues taking nirmatrelvir and ritonavir according to this schedule for a total of 5 consecutive days. Patient 2 shows an improvement in at least one symptom or biomarker of PASC.

Patient 3: Patient 3 takes an initial single dose of efalizumab via subcutaneous administration in the dosage amount of 0.7 mg. On the same day, Patient 3 begins taking sofosbuvir orally in the dosage amount of 400 mg. Patient 3 takes this dosage amount of sofosbuvir once a day for a period of 12 weeks. Patient 3 continues to take additional doses of efalizumab via subcutaneous administration weekly in the dosage amount of 1 mg. Patient 3 shows an improvement in at least one symptom or biomarker of PASC.

Patient 4: Patient 4 takes a loading dose of abciximab via intravenous infusion in the dosage amount of 0.25 mg. Patient 4 then receives a maintenance dose of abciximab via intravenous infusion in the dosage amount of 10 mcg/min. On the same day, Patient 4 begins taking sofosbuvir orally in the dosage amount of 400 mg. Patient 4 takes this dosage amount of sofosbuvir once a day for a period of 12 weeks. Patient 4 shows an improvement in at least one symptom or biomarker of PASC.

Patient 5: Patient 5 takes a standard dose of nirmatrelvir/ritonavir (Paxlovid™) via oral administration, i.e., 300 mg of nirmatrelvir and 100 mg of ritonavir. Patient 5 begins taking one standard dose of two tablets of nirmatrelvir (each 150 mg) and one tablet of ritonavir (100 mg) two times daily, in accordance with the package instructions. Specifically, Patient 5 takes one standard dose at the same time each morning and at the same time each evening for a period of five days. On the same day, Patient 5 receives intravenous administration of one dose of lenzilumab in the amount of 600 mg. Eight hours later, Patient 5 receives intravenous administration of a second dose (containing 600 mg) of lenzilumab. Patient 5 receives a final dose of lenzilumab (containing 600 mg) via intravenous administration eight hours later. Patient 5 shows an improvement in at least one symptom or biomarker of PASC.

Patient 6: Patient 6 takes a reduced dose of nirmatrelvir/ritonavir (Paxlovid™) via oral administration, i.e., 150 mg of nirmatrelvir and 100 mg of ritonavir. Patient 6 begins taking one reduced dose of one tablet of nirmatrelvir (150 mg) and one tablet of ritonavir (100 mg) two times daily, in accordance with the package instructions. Specifically, Patient 6 takes one reduced dose at the same time each morning and at the same time each evening for a period of five days. On the same day, Patient 6 receives administration subcutaneously of a dose of canakinumab in an amount of 150 mg. Patient 6 receives an additional dose of canakinumab in an amount of 150 mg every 8 weeks via subcutaneous administration. Patient 6 shows an improvement in at least one symptom or biomarker of PASC.

Patient 7: Patient 7 takes a standard dose of nirmatrelvir/ritonavir (Paxlovid™) via oral administration, i.e., 300 mg of nirmatrelvir and 100 mg of ritonavir. Patient 7 begins taking one standard dose of two tablets of nirmatrelvir (each 150 mg) and one tablet of ritonavir (100 mg) two times daily. Specifically, Patient 7 takes one standard dose at the same time each morning and at the same time each evening for a period of ten days. Two days later, Patient 7 takes a single dose of tixagevimab/cilgavimab (Evusheld™), administered via intramuscular injection (150 mg tixagevimab and 150 mg cilgavimab). Patient 7 continues to take a standard dose of nirmatrelvir/ritonavir via oral administration for eight days following the administration of the first dose of tixagevimab/cilgavimab. Patient 7 continues to receive a single dose of tixagevimab/cilgavimab administered via intramuscular injection once a month, beginning one month after receiving the first dose, for two additional consecutive months. Patient 7 takes a standard oral dose of nirmatrelvir/ritonavir, beginning two days before administration of each dose of tixagevimab/cilgavimab, and continues taking a standard dose of nirmatrelvir/ritonavir for eight days following administration of each dose of tixagevimab/cilgavimab. Patient 7 shows an improvement in at least one symptom or biomarker of PASC.

Patient 8: Patient 8 takes a standard dose of nirmatrelvir/ritonavir (Paxlovid™) via oral administration, i.e., 300 mg of nirmatrelvir and 100 mg of ritonavir. Patient 8 begins taking one standard dose of two tablets of nirmatrelvir (each 150 mg) and one tablet of ritonavir (100 mg) two times daily. Specifically, Patient 8 takes one standard dose at the same time each morning and at the same time each evening for a period of ten days. Two days later, Patient 8 takes a single dose of tixagevimab/cilgavimab (Evusheld™), administered via intramuscular injection (150 mg tixagevimab and 150 mg cilgavimab). Patient 8 continues to take a standard oral dose of nirmatrelvir/ritonavir for eight days following the administration of the first dose of tixagevimab/cilgavimab. Patient 8 shows an improvement in at least one symptom or biomarker of PASC.

Patient 9: Patient 9 takes a standard dose of nirmatrelvir/ritonavir (Paxlovid™) via oral administration, i.e., 300 mg of nirmatrelvir and 100 mg of ritonavir. Patient 9 begins taking one standard dose of two tablets of nirmatrelvir (each 150 mg) and one tablet of ritonavir (contains 100 mg) two times daily. Specifically, Patient 9 takes one standard dose at the same time each morning and at the same time each evening for a period of ten days. Two days later, Patient 9 takes a single dose of tixagevimab/cilgavimab (Evusheld™), administered via intramuscular injection (150 mg tixagevimab and 150 mg cilgavimab). Patient 9 continues to take a standard oral dose of nirmatrelvir/ritonavir for eight days following the administration of the first dose of tixagevimab/cilgavimab. Patient 9 continues to receive a single dose of tixagevimab/cilgavimab administered via intramuscular injection once a month, beginning one month after receiving the first dose, for five additional consecutive months. Patient 9 takes a standard oral dose of nirmatrelvir/ritonavir, beginning two days before administration of each dose of tixagevimab/cilgavimab, and continues taking a standard dose of nirmatrelvir/ritonavir for eight days following administration of each dose of tixagevimab/cilgavimab. Patient 9 shows an improvement in at least one symptom or biomarker of PASC.

Patient 10: Patient 10 takes a standard dose of nirmatrelvir/ritonavir (Paxlovid™) via oral administration, i.e., 300 mg of nirmatrelvir and 100 mg of ritonavir. Patient 10 begins taking one standard dose of two tablets of nirmatrelvir (each 150 mg) and one tablet of ritonavir (contains 100 mg) two times daily. Specifically, Patient 10 takes one standard dose at the same time each morning and at the same time each evening for a period of ten days. Two days later, Patient 10 takes a single dose of tixagevimab/cilgavimab (Evusheld™), administered via intramuscular injection (150 mg tixagevimab and 150 mg cilgavimab). Patient 10 continues to take a standard oral dose of nirmatrelvir/ritonavir for eight days following the administration of the first dose of tixagevimab/cilgavimab. Patient 10 continues to receive a single dose of tixagevimab/cilgavimab administered via intramuscular injection twice a month, beginning two weeks after receiving the first dose, for two additional consecutive months. Patient 10 takes a standard oral dose of nirmatrelvir/ritonavir, beginning two days before administration of each dose of tixagevimab/cilgavimab, and continues taking a standard dose of nirmatrelvir/ritonavir for eight days following administration of each dose of tixagevimab/cilgavimab. Patient 10 shows an improvement in at least one symptom or biomarker of PASC.

Patient 11: Patient 11 takes a standard dose of nirmatrelvir/ritonavir (Paxlovid™) via oral administration, i.e., 300 mg of nirmatrelvir and 100 mg of ritonavir. Patient 11 begins taking one standard dose of two tablets of nirmatrelvir (each 150 mg) and one tablet of ritonavir (contains 100 mg) two times daily. Specifically, Patient 11 takes one standard dose at the same time each morning and at the same time each evening for a period of ten days. Five days later, Patient 11 takes a single dose of tixagevimab/cilgavimab (Evusheld™), administered via intramuscular injection (150 mg tixagevimab and 150 mg cilgavimab). Patient 11 continues to take a standard oral dose of nirmatrelvir/ritonavir for five days following the administration of the first dose of tixagevimab/cilgavimab. Patient 11 continues to receive a single dose of tixagevimab/cilgavimab administered via intramuscular injection once a month, beginning one month after receiving the first dose, for two additional consecutive months. Patient 11 takes a standard oral dose of nirmatrelvir/ritonavir, beginning five days before administration of each dose of tixagevimab/cilgavimab, and continues taking a standard dose of nirmatrelvir/ritonavir for five days following administration of each dose of tixagevimab/cilgavimab. Patient 11 shows an improvement in at least one symptom or biomarker of PASC.

Patient 12: Patient 12 takes a single dose of tixagevimab/cilgavimab (Evusheld™), administered via IM injection (150 mg tixagevimab and 150 mg cilgavimab). Five days later, Patient 12 takes a standard dose of nirmatrelvir/ritonavir (Paxlovid™) via oral administration, i.e., 300 mg of nirmatrelvir and 100 mg of ritonavir. Patient 12 begins taking one standard dose of two tablets of nirmatrelvir (each 150 mg) and one tablet of ritonavir (100 mg) two times daily. Specifically, Patient 12 takes one standard dose at the same time each morning and at the same time each evening for a period of ten days. Patient 12 continues to receive a single dose of tixagevimab/cilgavimab administered via intramuscular injection once a month, beginning one month after receiving the first dose, for two additional consecutive months. Patient 12 takes a standard oral dose of nirmatrelvir/ritonavir, beginning five days after administration of each dose of tixagevimab/cilgavimab. Patient 12 shows an improvement in at least one symptom or biomarker of PASC.

Patient 13: Patient 13 takes a standard dose of nirmatrelvir/ritonavir (Paxlovid™) via oral administration, i.e., 300 mg of nirmatrelvir and 100 mg of ritonavir. Patient 13 begins taking one standard dose of two tablets of nirmatrelvir (each 150 mg) and one tablet of ritonavir (100 mg) two times daily. Specifically, Patient 13 takes one standard dose at the same time each morning and at the same time each evening for a period of ten days. Three days later, Patient 13 takes a single dose of tixagevimab/cilgavimab (Evusheld™), administered via intramuscular injection (150 mg tixagevimab and 150 mg cilgavimab). Patient 13 continues to take a standard oral dose of nirmatrelvir/ritonavir for seven days following the administration of the first dose of tixagevimab/cilgavimab. Patient 13 continues to receive a single dose of tixagevimab/cilgavimab administered via intramuscular injection once a month, beginning one month after receiving the first dose, for two additional consecutive months. Patient 13 takes a standard oral dose of nirmatrelvir/ritonavir, beginning 3 days before administration of each dose of tixagevimab/cilgavimab, and continues taking a standard dose of nirmatrelvir/ritonavir for 7 days following administration of each dose of tixagevimab/cilgavimab. Patient 13 shows an improvement in at least one symptom or biomarker of PASC.

Patient 14: Patient 14 takes a standard dose of nirmatrelvir/ritonavir (Paxlovid™) via oral administration, i.e., 300 mg of nirmatrelvir and 100 mg of ritonavir. Patient 14 begins taking one standard dose of two tablets of nirmatrelvir (each 150 mg) and one tablet of ritonavir (100 mg) two times daily. Specifically, Patient 14 takes one standard dose at the same time each morning and at the same time each evening for a period of ten days. Three days later, Patient 14 takes a single dose of tixagevimab/cilgavimab (Evusheld™), administered via intramuscular injection (150 mg tixagevimab and 150 mg cilgavimab). Patient 14 continues to take a standard oral dose of nirmatrelvir/ritonavir for seven days following the administration of the first dose of tixagevimab/cilgavimab. Patient 14 continues to receive a single dose of tixagevimab/cilgavimab administered via intramuscular injection once a month, beginning one month after receiving the first dose, for two additional consecutive months. Patient 14 takes a standard oral dose of nirmatrelvir/ritonavir, beginning 3 days before administration of each dose of tixagevimab/cilgavimab, and continues taking a standard dose of nirmatrelvir/ritonavir for 7 days following administration of each dose of tixagevimab/cilgavimab. Patient 11 shows an improvement in at least one symptom or biomarker of PASC.

Patient 15: Patient 15 takes a standard dose of nirmatrelvir/ritonavir (Paxlovid™) via oral administration, i.e., 300 mg of nirmatrelvir and 100 mg of ritonavir. Patient 15 begins taking one standard dose of two tablets of nirmatrelvir (each 150 mg) and one tablet of ritonavir (contains 100 mg) two times daily. Specifically, Patient 15 takes one standard dose at the same time each morning and at the same time each evening for a period of ten days. Seven days later, Patient 15 takes a single dose of tixagevimab/cilgavimab (Evusheld™), administered via intramuscular injection (150 mg tixagevimab and 150 mg cilgavimab). Patient 15 continues to take a standard oral dose of nirmatrelvir/ritonavir for three days following the administration of the first dose of tixagevimab/cilgavimab. Patient 15 continues to receive a single dose of tixagevimab/cilgavimab administered via intramuscular injection once a month, beginning one month after receiving the first dose, for two additional consecutive months. Patient 15 takes a standard oral dose of nirmatrelvir/ritonavir, beginning seven days before administration of each dose of tixagevimab/cilgavimab, and continues taking a standard dose of nirmatrelvir/ritonavir for three days following administration of each dose of tixagevimab/cilgavimab. Patient 15 shows an improvement in at least one symptom or biomarker of PASC.

Patient 16: Patient 16 takes a single dose of tixagevimab/cilgavimab (Evusheld™), administered via intramuscular injection (150 mg tixagevimab and 150 mg cilgavimab). Three days later, Patient 16 takes a standard dose of nirmatrelvir/ritonavir (Paxlovid™) via oral administration, i.e., 300 mg of nirmatrelvir and 100 mg of ritonavir. Patient 16 begins taking one standard dose of two tablets of nirmatrelvir (each 150 mg) and one tablet of ritonavir (contains 100 mg) two times daily. Specifically, Patient 16 takes one standard dose at the same time each morning and at the same time each evening for a period of ten days. Patient 16 continues to receive a single dose of tixagevimab/cilgavimab administered via intramuscular injection once a month, beginning one month after receiving the first dose, for two additional consecutive months. Patient 16 takes a standard oral dose of nirmatrelvir/ritonavir, beginning three days after administration of each dose of tixagevimab/cilgavimab. Patient 16 shows an improvement in at least one symptom or biomarker of PASC.

Patient 17: Patient 17 takes a single dose of tixagevimab/cilgavimab (Evusheld™), administered via intramuscular injection (150 mg tixagevimab and 150 mg cilgavimab). Seven days later, Patient 17 takes a standard dose of nirmatrelvir/ritonavir (Paxlovid™) via oral administration, i.e., 300 mg of nirmatrelvir and 100 mg of ritonavir. Patient 17 begins taking one standard dose of two tablets of nirmatrelvir (each 150 mg) and one tablet of ritonavir (contains 100 mg) two times daily. Specifically, Patient 17 takes one standard dose at the same time each morning and at the same time each evening for a period of 10 days. Patient 17 continues to receive a single dose of tixagevimab/cilgavimab administered via IM injection once a month, beginning one month after receiving the first dose, for two additional consecutive months. Patient 17 takes a standard oral dose of nirmatrelvir/ritonavir, beginning seven days after administration of each dose of tixagevimab/cilgavimab. Patient 17 shows an improvement in at least one symptom or biomarker of PASC.

Patient 18: Patient 18 takes single dose of tixagevimab/cilgavimab (Evusheld™), administered via intramuscular injection (150 mg each of tixagevimab and cilgavimab). On the same day, Patient 18 begins taking 400 mg sofosbuvir orally. Patient 18 takes this dosage amount of sofosbuvir once a day for a period of 12 weeks. Patient 18 shows an improvement in at least one symptom or biomarker of PASC.

Patient 0: In this example, a patient was administered 3 treatments of Evusheld™, once a month for 3 consecutive months, doubling the dose after the first administration (according to FDA guidelines, after the FDA recommended the dose to be increased). The patient also received 3×10 days of Paxlovid™ treatment, administered for the 2 preceding days before each of the 3 Evusheld™ injections, and for the following 8 days afterwards. The patient showed significant improvements in multiple PASC symptoms.

Additional exemplary treatment methods include any of the foregoing embodiments as illustrated by Patients 0-18 above, but wherein sofosbuvir is administered in place of nirmatrelvir/ritonavir (Paxlovid™). Additional exemplary treatment methods include any of the foregoing embodiments, but wherein any antiviral agent, or pharmaceutically acceptable salt thereof, disclosed herein is administered in place of any of sofosbuvir, nirmatrelvir/ritonavir (or Paxlovid™), remdesivir, or any other antiviral agent used with Patients 0-18. Additional exemplary treatment methods include any of the foregoing embodiments, but wherein any antibody disclosed herein is administered in place of tixagevimab/cilgavimab (Evusheld™) or any other antibody used with Patients 0-18. Additional exemplary treatment methods include any of the foregoing embodiments, but wherein any of the combinations used with Patients 0-18 further comprises one or more additional active agents, as disclosed herein. It will be readily appreciated that other such substitutions can be made, using any one or more other of the pharmaceutical agents disclosed herein, to provide additional pharmaceutical combinations, which may be used in the disclosed methods, according to the invention.

### Example 2: Study Assessing a Disclosed Method or Combination to Treat a Subject with PASC

Purpose: To measure the efficacy of disclosed methods & combinations to treat subjects with PASC.

Eligibility: All subjects undergo collection of medical history and physical examination. A treatment group is established which includes individuals diagnosed with PASC. PASC is diagnosed in a subject according to diagnostic techniques known to those of skill, for example, by detection of SARS-CoV-2 virus (e.g., by real-time reverse transcriptase-polymerase chain reaction (RT-PCR) during a time period of at least 28 days after the first positive PCR test, or otherwise from the date selected as the date of infection; or is diagnosed from the subject's medical history, for example, because of the persistence or appearance of conditions(s), disease(s), syndrome(s), symptom(s), or disability(ies) following a SARS-CoV-2 infection that are most probably causally related to the SARS-CoV-2 infection. A control group is established which includes healthy subjects with no history of SARS-CoV-2 virus. Subjects will be over the age of 18 years. Subjects can withdraw from the study for any time, and for any reason. Subjects unwilling to participate in the study, or having a medical condition that contraindicates them for treatment with a disclosed method or combination will also be excluded from the study.

Study Design Overview: Subjects will be given a data logbook, or clinical app for their phone, or other means for recording incidence and severity of symptoms, in which they will record the number and severity of PASC symptoms e.g., fatigue, headache, dizziness, difficulty thinking or concentration (i.e., brain fog), change or loss in smell or taste, depression, anxiety, chest pain, joint pain, tachycardia, cough, skin lesions, kidney issues) they experience each day. Subjects will be contacted by mail, email, phone, and/or sent reminders through a clinical phone app every two days to review symptoms and protocol compliance.

Subjects will also be subjected to laboratory testing for diagnostic markers of PASC, including the measurement of serum concentrations of the PASC biomarkers albumin, CRP, ferritin, G-CSF, IFN-α, IL-1β, IL-6, IL-13, IL-17, IP-10, TNF-α, D-dimer, and hemoglobin. The serum concentrations of each PASC biomarker measured across the control subjects are averaged. The average of each PASC biomarker is used to establish a “healthy PASC biomarker profile” for later comparison.

Symptoms will be recorded for a suitable time period prior to treatment. Subjects in the treatment group will then be divided into the following groups:

| Group | Treatment |
|---|---|
| 1 | Standard of Care (Control treatment) |
| 2 | (First) antiviral agent |
| 3 | (First) antiviral agent + (first) antibody |
| 4 | (First) antiviral agent + (second) antiviral agent + (first) |
| | antibody |
| 5 | (First) antiviral agent + (first) antibody + (second) antibody |
| 6 | (First) antiviral agent + (second) antiviral agent + |
| | (first) antibody + (second) antibody |

In general, the dose and dosage of the antiviral agents and antibodies will be as disclosed herein, or in accordance with the package instructions or accepted medical guidelines, both of which will be known to those of skill in the art. Control subjects will not receive any treatment.

Observations and Measurements of the Study: Observations for efficacy will include evaluation of PASC symptoms (e.g., fatigue, headache, dizziness, difficulty thinking or concentration (i.e., brain fog), change or loss in smell or taste, depression, anxiety, chest pain, joint pain, tachycardia, cough, skin lesions, kidney issues). Any additional adverse effects will also be monitored. Patients will undergo laboratory testing to measure post-treatment serum concentrations of the PASC biomarkers albumin, CRP, ferritin, G-CSF, IFN-α, IL-1β, IL-6, IL-13, IL-17, IP-10, TNF-α, D-dimer, and hemoglobin.

Results: Results will show that prior to treatment, the PASC biomarker profile of treatment subjects differ significantly from the healthy PASC biomarker profile. Further, the PASC biomarker profile of each treatment subject correlates to their reported symptoms and to the severity of those symptoms. After treatment with a disclosed method or combination, the PASC biomarker profiles of treatment subjects will not significantly differ from the healthy PASC biomarker profile. Results will show that a disclosed method or combination reduces the incidence, eliminates the incidence, or reduces or severity of PASC symptoms.

### Example 3: In Vitro Efficacy of Disclosed Combinations to Treat PASC-Associated Cytopathy

The ability of a disclosed combination to prevent PASC-associated cell death or cytopathy, can be assessed in vitro via cell viability, using an assay format that utilizes luciferase to measure intracellular ATP as an endpoint. In brief, VeroE6 cells that are enriched for hACE2 expression are batched inoculated with SARS-CoV-2. Virus-inoculated cells are then added to assay-ready plates containing an antiviral agent or monoclonal antibody, or combination thereof, as described herein. Following a multi-day incubation, or at a time at which virus-induced cytopathic effect is 95% in the untreated, infected control conditions, cell viability is evaluated using Cell Titer Glo (Promega), according to the manufacturer's protocol, which quantitates ATP levels. Cytotoxicity of antiviral agent(s), monoclonal antibody(ies), and combinations thereof, is assessed in parallel non-infected cells. Test combinations of antiviral agents and antibodies are tested either alone or in the presence of the P-glycoprotein (P-gp) inhibitor CP-100356 at a concentration of 2 pM. The inclusion of CP-100356 is to assess if the test antiviral agents are being effluxed out of the VeroE6 cells, which have high levels of expression of P-glycoprotein. Percent effect at each concentration of test antiviral agent(s), monoclonal antibody(ies), or combination thereof is calculated based on the values for the no virus control wells and virus containing control wells on each assay plate. The concentration required for a 50% response (EC50) value is determined from these data using a 4-parameter logistic model. EC50 curves are fit to a Hill slope of 3 when >3 and the top dose achieves 50% effect. If cytotoxicity is detected at greater than 30% effect, the corresponding concentration data is eliminated from the EC50 determination.

For cytotoxicity plates, a percent effect at each concentration of antiviral agent(s), antibody(ies), or combination(s) thereof, is calculated based on the values for the cell-only control wells and hyamine-containing control wells on each assay plate. The CC50 value is calculated using a 4-parameter logistic model. A TI was then calculated by dividing the CC50 value by the EC50 value.

Results may show that disclosed combinations can effectively prevent PASC-associated cell death or cytopathy, which indicates that disclosed methods can reduce the incidence or severity of a PASC-associated cytopathic effect.

### Example 4: Adaptive Trial Design for Selecting Additional Pharmaceutical Compositions

An “adaptive” trial design mechanism may be utilized in the performance of clinical trials to measure the efficacy of a disclosed method or combination for treating subjects with PASC or a symptom of PASC. Without being bound by theory, an adaptive design is a design that allows modifications to the trial and/or statistical procedures of the trial after its initiation without undermining its validity and integrity. Adaptive designs therefore may make clinical trials more flexible by utilizing results accumulating in the trial to modify the course of the trial in accordance with pre-specified rules. Trials with an adaptive design are thus often more efficient, informative, and ethical than trials with a traditional fixed design since they often make better use of resources such as time and money, and may require fewer participants. An adaptive trial design can be applied across all phases of clinical research, from early-phase dose escalation to confirmatory trials.

Herein, an adaptive trial design may be used, in non-limiting examples, to select a combination of pharmaceutical agents for use in a disclosed method, to select a patient population for treatment, e.g., based on biomarkers, to select dosage forms, dose amounts, and/or a dosing regimen, to select when to initiate or terminate treatment, to select PASC symptoms which may be treated, and/or other such uses. An adaptive trial design, according to such methods as described herein, is expected to permit exploration of the combination space faster and more rigorously than a series of individual combination trials.

In a first step of an exemplary adaptive trial design useful in the practice of the invention, a specific combination of pharmaceutical agents is selected to be administered to subjects for treatment of PASC. In a second step, a clinical trial is initiated wherein the selected pharmaceutical combination is administered to the subjects. Clinical observations appropriate to the trial, e.g., of safety or efficacy, are made as subjects undergo treatment. Observations of efficacy may include reduction or elimination of PASC symptoms (e.g., fatigue, headache, sleep problems (e.g., insomnia), dizziness, difficulty thinking or concentration (i.e., brain fog), change or loss in smell or taste, depression, or anxiety). Adverse effects may be monitored. Observations are reviewed while the trial is underway, but before completion (as in a fixed-sample design). As observations are reviewed, trial design may be modified during the course of the trial, in accordance with the accumulating results and pre-specified rules, such as will be known to those of skill.

The foregoing description, for purposes of explanation, uses specific nomenclature to provide a thorough understanding of the invention. However, it will be apparent to one of skill that specific details are not required to practice the invention. The specific embodiments are presented for purposes of illustration, and not intended to be exhaustive or to limit the invention to the precise methods, compositions, formulations, or the like disclosed; many modifications and variations are possible in view of the teachings. Embodiments were chosen and described to best explain the principles of the invention and its practical applications, by way of specific examples, to enable those in the art to best utilize the invention and its embodiments with various modifications as are suited to the particular use contemplated, when such uses are beyond the specific examples disclosed. Accordingly, the scope of the invention shall be defined solely by the following claims and their equivalents.

The invention claimed is:

1. A method of treating a subject having post-acute sequelae of COVID-19 (PASC), the method comprising administering to a subject an effective amount of a combination of:
i) a first antiviral agent, or a pharmaceutically acceptable salt thereof;
ii) optionally, a second antiviral agent, or a pharmaceutically acceptable salt thereof;
iii) a first antibody; and
iv) optionally, a second antibody.

2. The method of claim 1, wherein the first antiviral agent is a protease inhibitor.

3. The method of claim 1, wherein the first antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

4. The method of claim 1, wherein the first antibody is a monoclonal antibody.

5. The method of claim 1, wherein the first antibody is an Immunoglobulin G (IgG) antibody.

6. The method of claim 1, wherein the first antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, Anti-Endothelin Receptor B Antibody (AER002), and durvalumab.

7. The method of claim 1, wherein the second antiviral agent is a protease inhibitor.

8. The method of claim 1, wherein the second antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

9. The method of claim 1, wherein the second antibody is a monoclonal antibody.

10. The method of claim 1, wherein the second antibody is an IgG antibody.

11. The method of claim 1, wherein the second antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, AER002, and durvalumab.

12. The method of claim 1, further comprising administering an effective amount of an additional active agent.

13. The method of claim 12, wherein the additional active agent is any of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, monoamine oxidase inhibitors, empathogens, entactogens, entheogens, psychedelics, dissociatives, nootropics, euphorics, sedatives, stimulants, and vitamins.

14. The method of claim 12, wherein the additional active agent is any of an uncoating inhibitor, entry inhibitor, protease inhibitor, immunostimulatory agent, latency reversal agent, reverse transcriptase, reverse transcriptase inhibitor, integrate inhibitor, nucleoside analogue, RNA polymerase inhibitor, viral uncapping agent, neuraminidase inhibitor, fusion inhibitor, interferon, nucleoside reverse transcriptase inhibitor, or non-nucleoside reverse transcriptase inhibitor.

15. The method of claim 12, wherein the additional active agent is an immunostimulatory agent, a COVID-19 vaccine, an additional agent having antiviral activity, or an additional antibody.

16. The method of claim 15, wherein the additional agent having antiviral activity is MIR 19® (siR-7-EM/KK-46), baicalein, peginterferon lambda, metformin, ursodeoxycholic acid (UCDA), TVGN-489, LAU-7b, sabizabulin, azithromycin, ivermectin, opanganib, baricitinib, mycophenolate, APN01, epigallocatechin gallate (EGCG), diphenhydramine, quercetin, Qing-Fei-Pai-Du-Tang (QFPDT), Lianhua Qingwen (LQ), Taiwan Chingguan Yihau (NRICM101), Jing Si herbal drink (JSHD), nicotine, lactoferrin, nattokinase, or ubiquinone.

17. The method of claim 1, wherein the first antiviral agent is nirmatrelvir, or a pharmaceutically acceptable salt thereof; sofosbuvir, or a pharmaceutically acceptable salt thereof; or ritonavir, or a pharmaceutically acceptable salt thereof.

18. The method of claim 1, wherein the first antiviral agent is sofosbuvir, or a pharmaceutically acceptable salt thereof.

19. The method of claim 1, wherein the first antiviral agent is nirmatrelvir, or a pharmaceutically acceptable salt thereof; and the second antiviral agent is ritonavir, or a pharmaceutically acceptable salt thereof.

20. The method of claim 1, wherein the first antibody is tixagevimab or cilgavimab.

21. The method of claim 1, wherein the first antibody is tixagevimab and the second antibody is cilgavimab.

22. The method of claim 1, wherein the first antiviral agent is nirmatrelvir, or a pharmaceutically acceptable salt thereof; the second antiviral agent is ritonavir, or a pharmaceutically acceptable salt thereof; the first antibody is tixagevimab; and the second antibody is cilgavimab.

23. The method of claim 1, wherein the first antiviral agent is sofosbuvir, or a pharmaceutically acceptable salt thereof; the first antibody is tixagevimab; and the second antibody is cilgavimab.

24. The method of claim 1, wherein the first antiviral agent and the second antiviral agent are administered in a single pharmaceutical composition, or wherein the first antibody and the second antibody are administered in a single pharmaceutical composition.

25. The method of claim 1, resulting in a reduction in the incidence or severity of at least one symptom of PASC.

26. The method of claim 25, wherein the symptom of PASC is a neurological symptom, a cardiovascular symptom, a pulmonary symptom, a musculoskeletal symptom, an endocrinological symptom, a dermal symptom, or a kidney symptom.

27. The method of claim 26, wherein the neurological symptom is selected from the group consisting of sleep disorders, chronic headaches, olfactory disorders, taste disorders, brain fog, memory loss, decreased concentration, depression, anxiety, post-traumatic stress disorder (PTSD), dizziness, vertigo, tinnitus, hearing loss, instability, delirium, hallucinations, small fiber neuropathy, postural tremor, chronic pain, neurodegeneration, and myalgia.

28. The method of claim 26, wherein the cardiovascular symptom is selected from the group consisting of nonspecific chest pain, tightness in the chest, palpitations, tachycardia, conduction disturbances, rhythm disorders, orthostatic hypotension, vasovagal syncope, postural orthostatic tachycardia syndrome (POTS), phlebitis, and thrombophlebitis.

29. The method of claim 26, wherein the pulmonary symptom is selected from the group consisting of dyspnea, persistent cough, wheezing, worsening of asthma, decreased pulmonary diffusion capacity, persistent abnormal imaging findings, pleuritis, cough, sleep apnea, and sore throat.

30. The method of claim 26, wherein the musculoskeletal symptom is selected from the group consisting of arthritis, back pain, abnormal gait and joint pain.

31. The method of claim 26, wherein the endocrinological symptom is selected from the group consisting of impaired glucose metabolism, subacute thyrotoxicosis, Hashimoto's disease, Graves' disease, and dyslipidemia.

32. The method of claim 26, wherein the dermal symptom is selected from the group consisting of eruption, urticaria, skin lesion, telogen effluvium, nail changes, erythema, and trichodynia.

33. The method of claim 26, wherein the kidney symptom is selected from the group consisting of decreased globural filtration rate, urinary incontinence, and microscopic hematuria.

34. The method of claim 25, wherein the symptom of PASC is selected from the group consisting of fever, chills, muscle ache, body ache, chest pain, stomach pain, nausea, vomiting, diarrhea, gastroesophageal reflux, stress, swelling, bleeding, weight loss, weight gain, abdominal pain, constipation, pain in extremities, paresthesia, peripheral edema, itchiness, lymphadenopathy, and edema.

35. The method of claim 25, wherein the incidence of a symptom of PASC is reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%, as compared to the incidence of the symptom prior to treatment with the method.

36. The method of claim 25, wherein the severity of a symptom PASC is reduced by at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 99%, as compared to the severity of the symptom prior to treatment with the method.

37. The method of claim 1, resulting in the serum concentration of a PASC biomarker returning to within about 50%, 40%, 30%, 20%, 10%, 5%, or 1% of a baseline concentration.

38. The method of claim 37, wherein the PASC biomarker is a neurological biomarker.

39. The method of claim 38, wherein the neurological biomarker is GDNF, GFAP, NGF-β, NFL, NT-3, or pGFAP/pNFL.

40. The method of claim 37, wherein the PASC biomarker is a cardiovascular biomarker.

41. The method of claim 40, wherein the cardiovascular biomarker is Ang-2, Col1A2, Col3A1, D-dimer, ESR, ET-1, Factor VIII:C, Hemoglobin, MMP-1, MMP-9, MPO, NO, PDGF-BB, SICAM-1, sTM, sVEGFR, SVCAM-1, VEGF, VWF:Ag, or VWF:pp.

42. A pharmaceutical kit for treating a subject having post-acute sequelae of COVID-19 (PASC), comprising:
i) a first antiviral agent, or a pharmaceutically acceptable salt thereof;
ii) optionally, a second antiviral agent, or a pharmaceutically acceptable salt thereof;
iii) a first antibody; and
iv) optionally, a second antibody.

43. The kit of claim 42, wherein the first antiviral agent is a protease inhibitor.

44. The kit of claim 42, wherein the first antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

45. The kit of claim 42, wherein the first antibody is a monoclonal antibody.

46. The kit of claim 42, wherein the first antibody is an Immunoglobulin G (IgG) antibody.

47. The kit of claim 42, wherein the first antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, Anti-Endothelin Receptor B Antibody (AER002), and durvalumab.

48. The kit of claim 42, wherein the second antiviral agent is a protease inhibitor.

49. The kit of claim 42, wherein the second antiviral agent is selected from the group consisting of ritonavir, nirmatrelvir, remdesivir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, molnupiravir, GC376, arbidol (umifenovoir), SSAA09E2, ribavarin, favilavir, oseltamivir, zanamivir, abacavir, stavudine, valganciclovir, ganciclovir, cidofovir, entecavir, amivudine, maraviroc, azidothymidine, nelfinavir, dolutegravir, olsetamivir, ensitrelvir (Xocova), simnotrelvir, bemnifosbuvir (AT-527), deuremidevir (VV116), azvudine, sofosbuvir, obeldesivir (GS-5245), acyclovir, and pharmaceutically acceptable salts thereof.

50. The kit of claim 42, wherein the second antibody is a monoclonal antibody.

51. The kit of claim 42, wherein the second antibody is an IgG antibody.

52. The kit of claim 42, wherein the second antibody is selected from the group consisting of tixagevimab, cilgavimab, pembrolizumab, nivolumab, bevacizumab, ocrelizumab, rituximab, daratumumab, pertuzumab, trastuzumab, infliximab, tocilizumab, atezolizumab, tositumomab, olaratumab, rituximab, basiliximab, ibritumomab tiuxetan, cetuximab, natalizumab, panitumumab, ranibizumab, eculizumab, ofatumumab, belimumab, ipilimumab, pertuzumab, raxibacumab, obinutuzumab, siltuximab, ramucirumab, vedolizumab, alemtuzumab, necitumumab, dinutuximab, elotuzumab, reslizumab, bezlotoxumab, obiltoxaximab, avelumab, sotrovimab, bebtelovimab, casivirimab, imdevimab, etesevimab, bamlanivimab, amubarvimab, regdanvimab, romlusevimab, adintrevimab, 002-S21F2, AZD3152, lenzilumab, ravulizumab, canakinumab, adalimumab, sarilumab, ronapreve, vilobelimab, casirivimab, sarilumab, leronlimab, golimumab, tabalumab, veltuzumab, mepolizumab, secukinumab, evolocumab, blinatumomab, adotrastuzumab, brentuximab, ipilumumab, denosumab, ustekinumab, catumaxomab, efalizumab, toitumomab-1131, palivizumab, basilixumab, daclizumab, abciximab, murononomab, certolizumab, benralizumab, dupilumab, omalizumab, muromonab-CD3, AER002, and durvalumab.

53. The kit of claim 42, further comprising an additional active agent.

54. The kit of claim 53, wherein the additional active agent is any of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, monoamine oxidase inhibitors, empathogens, entactogens, entheogens, psychedelics, dissociatives, nootropics, euphorics, sedatives, stimulants, and vitamins.

55. The kit of claim 53, wherein the additional active agent is any of an uncoating inhibitor, entry inhibitor, protease inhibitor, immunostimulatory agent, latency reversal agent, reverse transcriptase, reverse transcriptase inhibitor, integrate inhibitor, nucleoside analogue, RNA polymerase inhibitor, viral uncapping agent, neuraminidase inhibitor, fusion inhibitor, interferon, nucleoside reverse transcriptase inhibitor, or non-nucleoside reverse transcriptase inhibitor.

56. The kit of claim 53, wherein the additional active agent is an immunostimulatory agent, a COVID-19 vaccine, an additional agent having antiviral activity, or an additional antibody.

57. The kit of claim 56, wherein the additional agent having antiviral activity is MIR 19® (siR-7-EM/KK-46), baicalein, peginterferon lambda, metformin, ursodeoxycholic acid (UCDA), TVGN-489, LAU-7b, sabizabulin, azithromycin, ivermectin, opanganib, baricitinib, mycophenolate, APN01, epigallocatechin gallate (EGCG), diphenhydramine, quercetin, Qing-Fei-Pai-Du-Tang (QFPDT), Lianhua Qingwen (LQ), Taiwan Chingguan Yihau (NRICM101), Jing Si herbal drink (JSHD), nicotine, lactoferrin, nattokinase, or ubiquinone.

58. The kit of claim 42, wherein the first antiviral agent is nirmatrelvir, or a pharmaceutically acceptable salt thereof; sofosbuvir, or a pharmaceutically acceptable salt thereof, or ritonavir, or a pharmaceutically acceptable salt thereof.

59. The kit of claim 42, wherein the first antiviral agent is sofosbuvir, or a pharmaceutically acceptable salt thereof.

60. The kit of claim 42, wherein the first antiviral agent is nirmatrelvir, or a pharmaceutically acceptable salt thereof; and the second antiviral agent is ritonavir, or a pharmaceutically acceptable salt thereof.

61. The kit of claim 42, wherein the first antibody is tixagevimab or cilgavimab.

62. The kit of claim 42, wherein the first antibody is tixagevimab and the second antibody is cilgavimab.

63. The kit of claim 42, wherein the first antiviral agent is nirmatrelvir, or a pharmaceutically acceptable salt thereof; the second antiviral agent is ritonavir, or a pharmaceutically acceptable salt thereof; the first antibody is tixagevimab; and the second antibody is cilgavimab.

64. The kit of claim 42, wherein the first antiviral agent is sofosbuvir, or a pharmaceutically acceptable salt thereof; the first antibody is tixagevimab; and the second antibody is cilgavimab.

65. The kit of claim 42, wherein the first antiviral agent and the second antiviral agent are co-formulated as a single pharmaceutical composition, or wherein the first antibody and the second antibody are co-formulated as a single pharmaceutical composition.

66. Use of the pharmaceutical kit of claim 42 for treating PASC.

67. Use of the pharmaceutical kit of claim 42 for the manufacture of a medicament for treating PASC.
